(19) United States
(12) Patent Application Publication
(10) Pub. No.: US 2009/0082314 A1
(43) Pub. Date: Mar. 26, 2009

(54) Targeting Prodrugs for the Treatment of Gastrointestinal Diseases

(75) Inventors: John Francis Gilmer, Dublin (IE); Juan Francisco Marquez, Malaga (ES); Dermot Kelleher, Dublin (IE)

Correspondence Address: FOLEY & LARDNER LLP, 975 PAGE MILL ROAD, PALO ALTO, CA, 94304, US

(73) Assignees: The Provost Fellows and Scholars of the College of the; Holy and Undivided trinity of Queen Elizabeth near Dublin

(21) Appl. No.: 12/163,853

(22) Filed: Jun. 27, 2008

Related U.S. Application Data
(60) Provisional application No. 60/947,104, filed on Jun. 29, 2007.

(51) Int. Cl. A61K 31/655; C09B 56/00; C09B 29/00; A61K 31/573; A61P 35/00; A61P 29/00; C07K 16/00

(52) U.S. Cl. 514/150; 534/657; 534/660; 530/388.1; 514/179; 514/180; 514/174

(57) ABSTRACT

Provided herein are compounds, compositions and methods for decreasing NFκB DNA-binding activity in a patient comprising administering of a therapeutically effective amount of a compound or composition of the application to the patient to reduce, alleviate or treat various gastrointestinal diseases, such as inflammatory bowel disease (IBD).

This application claims the benefit of U.S. Provisional Application No. 60/947,104, filed Jun. 29, 2007, entitled “Targeting Prodrugs for the Treatment of Gastrointestinal Diseases” the disclosure of which is incorporated herein in its entirety.

### FIELD OF THE INVENTION

The present application relates to compounds and pharmaceutical compositions that may be used for the treatment of gastrointestinal diseases, including, but not limited to inflammatory diseases, such as Inflammatory Bowel Disease (IBD).

### BACKGROUND OF THE INVENTION

Drug targeting may be defined as the delivery of a drug to a specific organ, tissue or cell population (Schreier 2001). Still in its infancy, this field offers the prospect of enhancing the efficacy of drug treatment while reducing systemic impact or side effects. Despite the promise of this approach, and ongoing efforts, there have been few successful examples to date due in part to limited understanding of the basic factors underlying drug transport and the expression of potential targeting vectors. Chemical drug targeting involves the deliberate modification of a drug structure (usually bioreversibly) causing it to accumulate in a target tissue; site-specific release from the prodrug is triggered by a chemical or enzymatic condition not present elsewhere in the body.

The colon is an important challenge to the validity of the drug targeting approach, as conditions in the colon are largely similar to those prevailing elsewhere in the gastrointestinal (GI) system, and the luminal pH gradient through the GI tract is too gradual for effective local drug release on strictly chemical grounds (Bauer, 2001). On the other hand, the colon is an important drug target for the treatment of pathologies of the colon itself, such as inflammatory bowel disease (IBD) and colon cancer, and for the relief of the chronic constipation that accompanies opioid drug treatment. The colon is also important as a potential portal site for peptide and protein drugs that are not absorbed from other regions of the GI tract or are too unstable in the presence of duodenal proteases to be released there (Saffran, 1986; Bai, 1995).

One key difference between the colon and small intestine that might be exploited as a vector for site-specific drug release is the luxuriant microflora of the former. The human GI system is home to 400-500 species of bacteria with a total live population of 10¹⁴ organisms. This is remarkable when compared with the 10¹³ eukaryotic cells that make up the human body. The GI tract has a steadily increasing bacterial concentration gradient on descending from the stomach through the small intestine, followed by an enormous increase at the colon. The bacterial concentration in the small intestine is typically 10³-10⁴ CFU ml⁻¹ whereas the concentration in the colon is 10¹¹-10¹² CFU ml⁻¹ and one third of fecal dry weight consists of bacteria (Moore and Holdeman, 1974 and 1975; Simon and Gorbach, 1984). These organisms fulfill their energy needs by fermenting undigested materials entering from the small intestine (particularly polysaccharides) and have for this purpose evolved an elaborate array of enzymes such as azoreductase, glucosidase, β-glucuronidase, β-xylosidase, nitroreductase, galactosidase and deaminase (Scheline, 1973).

This abrupt increase in bacterial enzyme expression has been investigated as a means of targeting drugs to the colon, especially those for the treatment of IBD. One successful outcome of these endeavours has been the development of azo-based prodrugs of 5-amino salicylic acid (5-ASA) 1 (Scheme 1), which because of their hydrophilicity and polarity pass through the GI system intact before releasing their 5-amino salicylic acid ‘payload’ upon reduction of the azo linker by azoreductases associated with colonic microflora. Several drugs based on this concept, such as ipsalizide, balsalazide 2 (Chan et al. 1983), sulphasalazine (the prototype) and olsalazide 3 (Willoughby et al., 1982), are in clinical use for the treatment of IBD (Green, 1998).

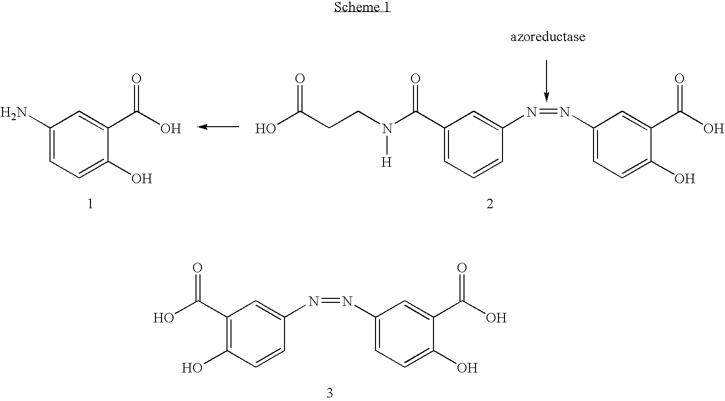

The targeting of other drug types, such as the anti-inflammatory steroids to the colon has been less successful (Sinha & Kumria 2001). Paradoxically, the need for appropriate systems in these cases is more pressing because steroids have multiple systemic side effects when administered orally due to their ready absorption from the stomach and small intestine. Chronic inflammatory bowel disease comprises two major disorders, namely ulcerative colitis and Crohn's disease. Both of these conditions produce significant morbidity in the form of diarrhea, weight loss and potentially serious and life-threatening complications. The incidence in Europe is estimated at up to 80 million/year (Logan, 1998), while the absolute incidence in the United States is believed to be about one million, with 15-30,000 new cases reported annually (DiPirio and Bowden, 1997). It is therefore unsurprising that intense efforts have been made both to unravel the underlying etiology of IBD and develop new therapies, but also to improve existing pharmacotherapy. These efforts have been restricted to 5-ASA derivatives and steroids.

The efforts to target steroids to the colon have been especially numerous. Technologies that have been investigated include: biodegradable polymers (Basit, 2000), time release systems, coating with pH sensitive materials, gastrointestinal pressure controlled release (Hu et al., 1998) and chemical drug targeting.

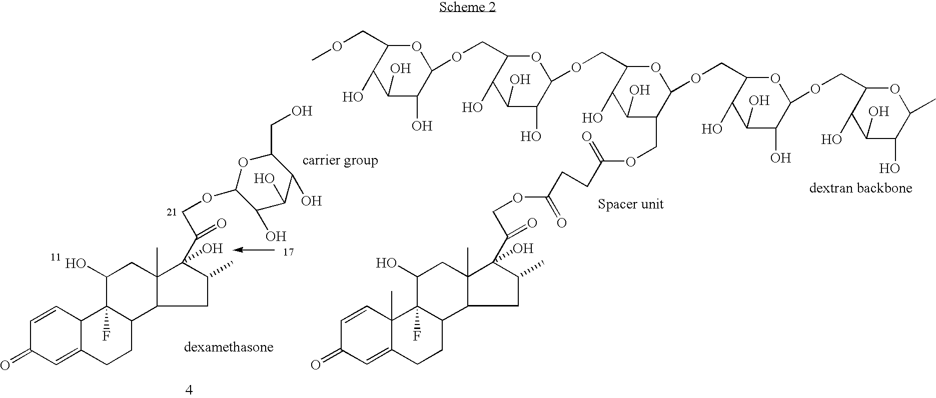

Approaches belonging to the latter class have revolved around the attachment to steroids of hydrophilic carriers that might act as substrates for bacterial polysaccharide processing enzymes (Scheme 2). Examples of carriers investigated include: glycosides (e.g. 4, References a-g); glucuronides (Haeberlin, 1993; Cui 1994); poly-(L-aspartic acid) derivatives (Leopold, 1995); α, β and γ-cyclodextrin conjugates (Yano, 2002); polymer conjugates (e.g. 5, McLeod et al., 1994). Although some of these designs are promising, they have thus far met with limited success, due in part to the widespread distribution of glycosidases in the GI tract (resulting in non-site specific release; Sinha & Kumria, 2001), and in some instances, because of slow release characteristics in the colon (up to 12 hours for some polysaccharides; Yang, 2002). The use of the successful azoreductase approach has thus far been restricted to drugs, such as 5-ASA, that bear a primary aromatic amine.

### SUMMARY OF THE INVENTION

Provided herein is a novel strategy for drug-targeting hydroxyl-bearing compounds to the colon. The innovative strategy for targeting hydroxyl-bearing drugs to the colon has been designed to overcome the design flaws in the glycosidase targeting approach. One method for achieving the site-specific delivery of hydroxyl-bearing drugs is schematically presented in Scheme 3. In one aspect, the drug is selected from the group that includes an anti-inflammatory drug, an anti-cancer drug, an imaging agent, particularly as used in the imaging of colon diseases; a vaccine, an antigen, an anti-infective drug, a peptide, an antisense molecule and a protein.

In one aspect of the present application, the method provides an evaluation of anti-inflammatory effects of candidate compounds in a mouse model of IBD. In another aspect, the method provides insights into the GI permeability of the azo-compounds and their ability to pass through the GI tract.

In the present application, the inventors discovered a need for compounds, such as the prodrugs disclosed herein, and compositions that are effective as selective agents for decreasing NFκB DNA-binding activity in a patient. In one aspect, there is provided a method comprising administering a therapeutically effective amount of a compound or composition effective to reduce, alleviate or treat various gastrointestinal diseases, including inflammatory bowel disease (IBD).

In one aspect, the drug is linked via an ester group to a carrier group, which is connected by an azo-bond to a second carrier group. The carrier groups may be directly attached to the azo group or indirectly attached to the azo group. As provided herein, the carrier groups are designed to maximally suppress absorption from stomach and upper intestine. The method exploits the selective reduction of an azo-linker in the colon, releasing a chemically unstable, latent prodrug that subsequently undergoes cyclization, such as lactamization, that liberates the drug payload, such as a steroid. In certain compounds, the cyclization reaction is substantially spontaneous. The overall effect of the design is to make the biologically stable or robust ester group, connecting the drug to the carrier group, chemically vulnerable under conditions found only in the colon.

Generally the prodrugs of the present application are referred to as “carrier-drug.” The “carrier” can comprise compounds such as 5-ASA or para-aminobenzoic acid (PABA). When the carrier has a therapeutic effect, such as 5-ASA, the prodrug is generally referred to as a “mutual prodrug.” Such mutual prodrugs can be referred to herein as 5-ASA-drug, wherein the drug can be any appropriate therapeutic agent, including those disclosed herein. Such mutual prodrugs can include, but are not limited to, 5-ASA-ciprofloxacin, 5-ASA-bevacizumab, 5-ASA-prednisolone, 5-ASA-5-ASA, etc. When the carrier does not have a therapeutic effect, such as PABA, the compound can be simply referred to as a “prodrug.” Such prodrugs can be referred to herein as PABA-drug, such as, for example PABA-ciprofloxacin, PABA-bevacizumab, PABA-prednisolone, PABA-5-ASA, etc.

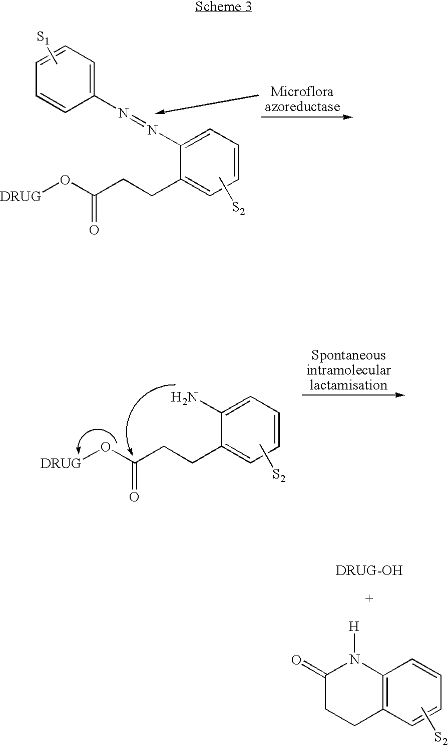

It was determined that the physicochemical characteristics of the prodrug can be optimized for gastrointestinal penetration to the colon by varying the nature of the compound, including the substituents S₁ and S₂, wherein one or more of the substituents S₁ and S₂ on the aryl ring may be employed, as disclosed herein.

Azo bond reduction proceeds readily because it is based on the promiscuity of the azoreductases present in the colon with respect to substrate, as evidenced by their ability to efficiently reduce substrates as diverse as ipsalazide, in which the carrier group is p-amino hippurate, balsalzide (p-aminobenzoyl-β-alanine carrier), sulfasalazine (sulfapyridine carrier), sterically bulky PAF antagonists (Carceller et al., 2001), 9-aminocamphothecin (Sakuma et al., 2001) and 5-ASA-N-methacrylamide, acryloyloxyethyl and acryloylamido copolymers (e.g. Van den Mooter et al., 1994). The presence of the vast microflora in the bowel causes a change in redox potential from −67±90 in the distal small bowel to −415±72 in the right colon (Wilding et al. 1994). The relative contribution of enzymatic and chemical reduction in the release of azo drugs is not well understood.

The cyclization or ring closure is dependent on nucleophilic attack by the amine group, such as the aniline amino group on the ester. An example of such ester is a steroidal ester. Although anilines possess low nucleophilicity (compared with aliphatic amines) the high effective molarity of the intramolecular arrangement will promote sufficiently rapid ring closure. Reports on the intramolecular aminolysis of esters by aniline, at roughly comparable conditions, have been described. Kirby et al. (1979) studied the spontaneous cyclization of methyl 3-(2-aminophenyl)propionate, finding that at neutrality (roughly the pH of the colon) and 39° C., the reaction proceeds with an apparent first-order rate constant of 2×10⁻⁴ sec⁻¹, corresponding to a first-order half-life of 57 min. Fife and Duddy (1983) reported the ring closure of methyl- and trifluoroethyl (2-aminophenyl)acetate (yielding the indolone) to follow similar kinetics under mildly basic conditions (pH 7-8). These half-lives are ideal for drug release in the colon, where residence time tends be rather extended.

The ester linkage in the compounds of the present application is highly stable under conditions found in the GI tract. Esterase activity in the lumen of the GI tract is restricted to the pancreatic serine proteases, which exhibit residual esterase activity towards a limited number of substrates, generally esters of aromatic amino acids. Steroidal 21-esters (see Scheme 2 above for numbering), for example, have been shown to be robust in simulated intestinal fluid models. For example Fleisher et al. (1986) reported a first-order rate constant of 0.003 min⁻¹ for the hydrolysis of hydrocortisone-21-succinate in rat intestinal perfusate. This figure corresponds to a half-life of 12 h which is significantly longer than the expected transit time to the colon. Jhunjhunwala (1981) reported the disappearance of less than 5% of hydrocortisone-21-succinate after 24 h at pH 7 (45° C.).

Gastrointestinal absorption is a function of molecular weight, lipophilicity and polarity; in general, polar, hydrophilic molecules are not well absorbed. Among the possibilities for variation at the carrier group comprising a substituent represented by S₁, for example, are azo linkage linking a carrier, including but not limited to 5-ASA, and a steroid, thereby generating a mutual prodrug of a drug, such as a steroid. Such mutual prodrugs may provide ideal or favourable physicochemical characteristics for passage through the intestine because of mass, polarity and hydrophilicity.

The sterically hindered steroidal esters disclosed in the present application are much less vulnerable towards hydrolysis than hydrocortisone-21-succinate (see Jhunjhunwala, 1981). Furthermore, the ester may also be attached on one of several different hydroxyl groups on the steroid nucleus. For example, without being bound by any theory proposed herein, the esters placed at the highly hindered 11β axial hydroxyl group (Scheme 2) are more stable than the 21-esters.

The present application discloses novel systems and compounds that are capable of engaging in a two-step process for releasing drugs, such as anti-inflammatory steroids, that target the colon. The present application also discloses the synthesis of a variety of compounds and their derivatives, including steroidal compounds (e.g. esters of hydrocortisone, dexamethasone or budesonide), nitroimidazoles (e.g. metronidazole), antibiotics, (e.g. quinolines such as nalidixic acid, fluoroquinolones, such as ciprofloxacin or levofloxacin, aminoglycosides, such as amikacin, gentamicin, kanamycin, neomycin, netilmicin, paromomycin, streptomycin, tobramycin and apramycin), chemotherapeutics (e.g. leucovorin, topotecan, irinotecan, methotrexate), and antibodies, (e.g. bevacizumab, cetuximab, panitumumab, infliximab). The present application further discloses the measurement of ring closure kinetics, in vitro screening for intestinal stability using enzymes and intestinal perfusates, drug release in vitro in the presence of colonic bacteria, and therapeutic efficacy, for example, anti-inflammatory efficacy, in appropriate animal models. In one aspect, a series of esters of the anti-inflammatory steroids hydrocortisone, dexamethasone and budesonide at the 21-, 11- and 17 positions are prepared as prodrugs. In another aspect, the application discloses the synthesis of various azo-derivatives of the amino esters.

The following Scheme provides representative biologically active agents and drugs, including anti-tumor agents (anthramycin, a sansalvamide derivative, capecitabine), COX-2 inhibitors (resveratrol, curcumin, meloxicam, tenoxicam, piroxicam), antibiotic agents (metronidazole), immunosuppressant agents (cyclosporine) and chemoprotective effect agents (URSO) that may be prepared and employed in the present application:

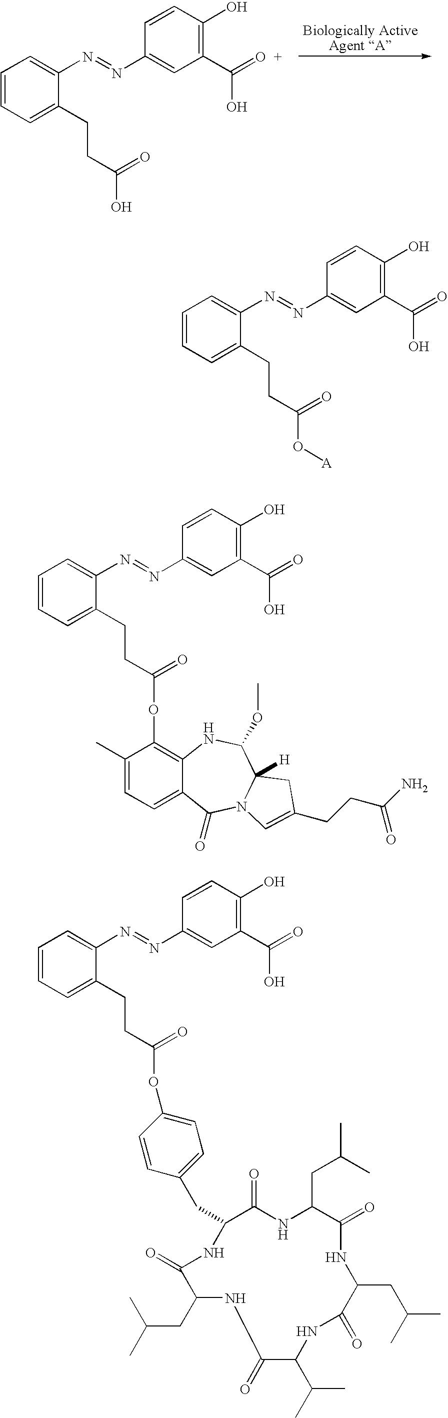

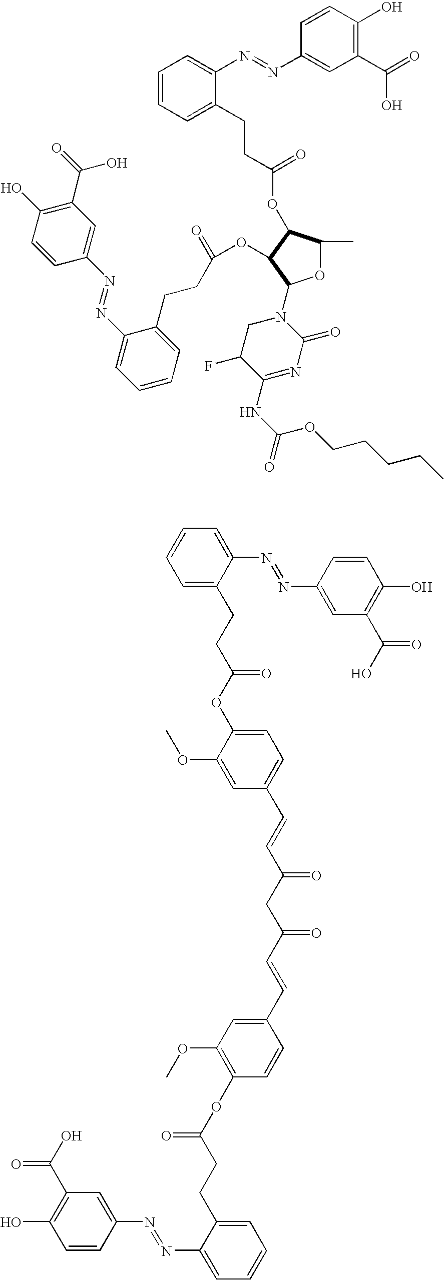

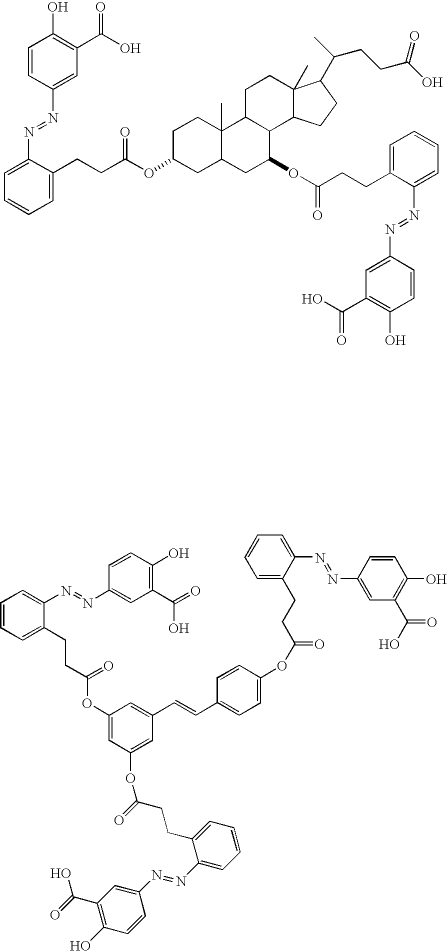

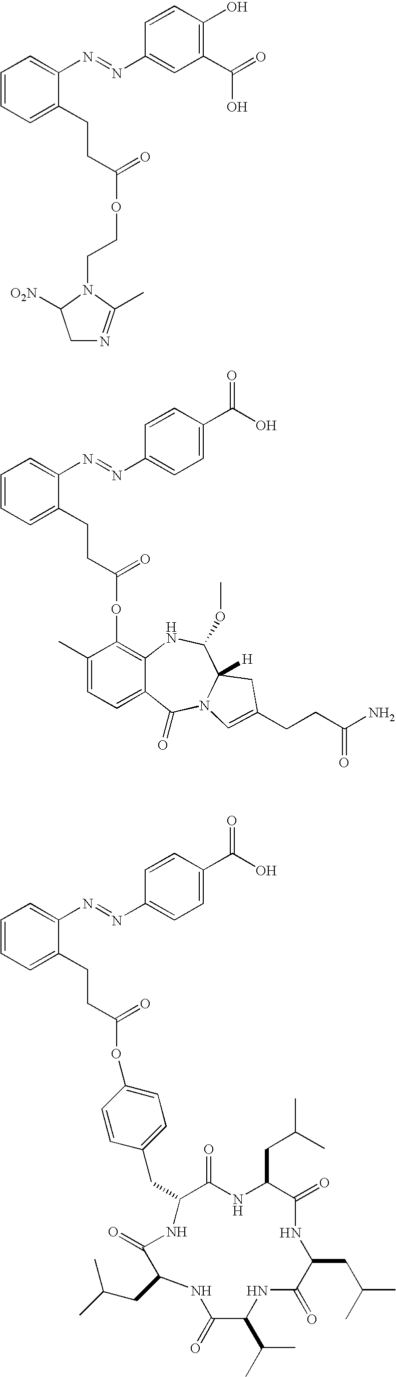

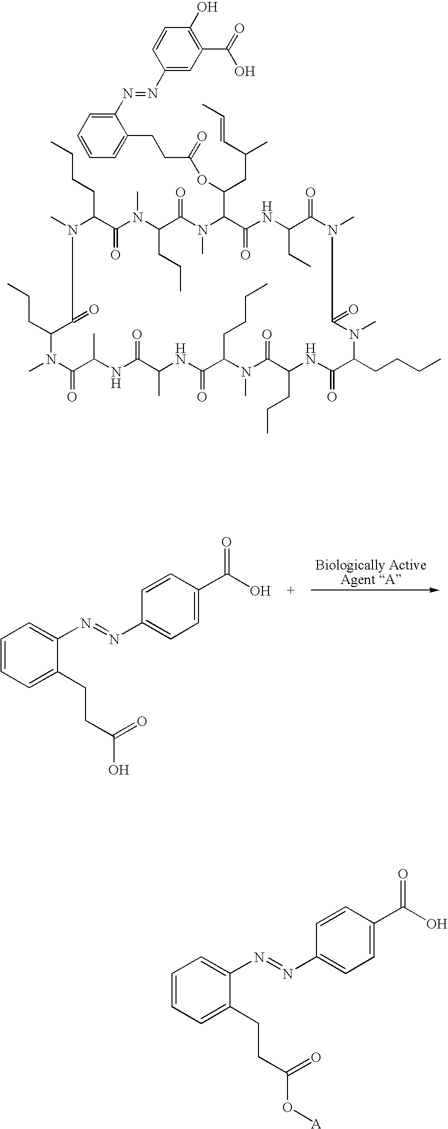

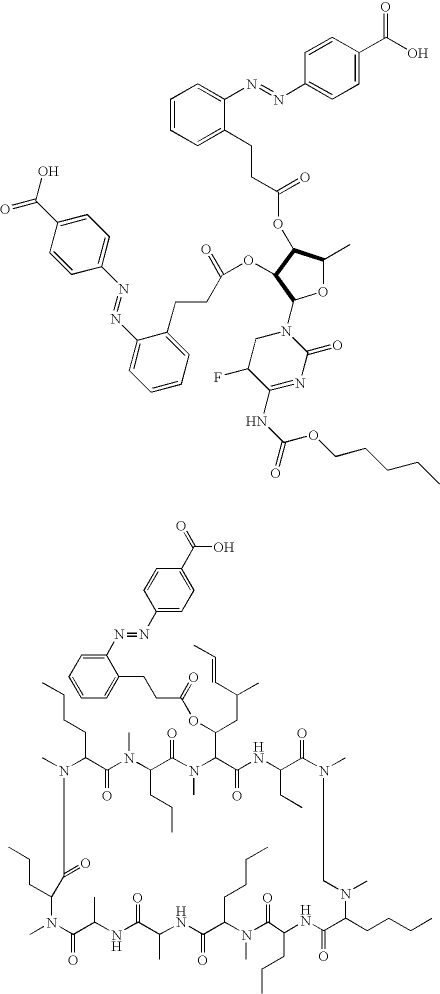

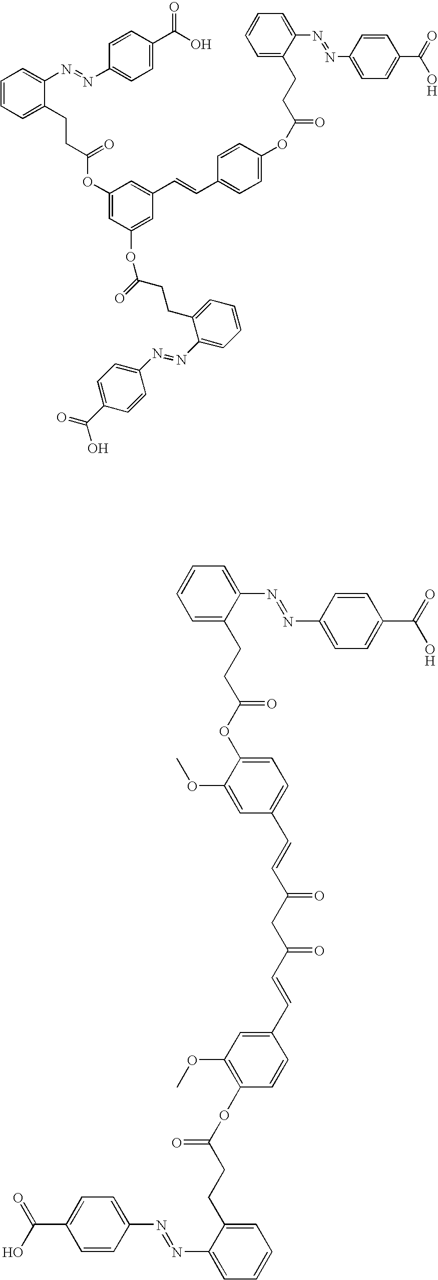

In another aspect, there is provided analytical methods for the determination or estimation of the rate of ring closure of the amino intermediate compounds. Also provided herein are methods for estimating the rate of ring closure and determining its dependence on mesomeric effects due to substitution in the carrier group, such as the aniline derivatives. In yet another aspect of the application, there is provided a method for measuring the stability of the compounds under aqueous conditions (at pH 1-8), in the presence of pancreatic serine proteases (in vitro) and other enzymes present in the gut and, in the presence of rat intestinal fluid.

In another aspect, there is provided a method for estimating the rate of reduction of these compounds in the presence of microflora in vitro. In yet another aspect, there is provided a method for estimating the in vivo efficacy of the compounds using a mouse model of colonic inflammation. In another aspect, there is provided a method for measuring the permeability of the compounds as provided herein, using rat perfusion models, which allows the assessment of penetration through the GI tract. For potential colon-targeting, compounds of negligible permeability are employed.

### BRIEF DESCRIPTION OF THE FIGURES

FIG. 1 is a graph of the concentration of prodrug A versus time at pH 8 (37° C.).

FIG. 2 is a graph of the natural log of the prodrug A concentration versus time at pH 8 (37° C.).

FIG. 3 is a graph of the concentration of the prodrug A versus time at pH 7.4 (37° C.).

FIG. 4 is a graph of the natural log of the prodrug A concentration versus time in at pH 7.4 (37° C.).

FIG. 5 is a profile of the lactamization of aminoprodrug B at pH 9 (37° C.) showing the disappearance of the prodrug and appearance of cortisone and the quinolone.

FIG. 6 is a graph of the natural log of the aminoprodrug B concentration versus time at pH 9 (37° C.).

FIG. 7 is a profile of the lactamization of amino prodrug B at pH 8 (37° C.).

FIG. 8 is a graph of the natural log of the concentration of aminoprodrug B versus time at pH 8.

FIG. 9 is a profile of the lactamization of aminoprodrug B in PBS at pH 9 (37° C.) showing the disappearance of the prodrug and appearance of hydrocortisone and quinolone.

FIG. 10 is a graph of the natural log of the concentration of aminoprodrug B at pH 7.4 (37° C.).

FIG. 11 is a profile of the body weight loss percentage of healthy mice (Untreated) and dextran sodium sulphate-induced colitis (DSS-induced colitis) mice treated at 5 mg/Kg dosage.

FIG. 12 is a profile of the Disease Activity Index (DAI) score from colon segment of DSS-induced colitis mice treated at 5 mg/Kg dosage.

FIG. 13 is a profile of colon length of DSS-induced colitis mice treated at 5 mg/Kg dosage.

FIG. 14 is a profile of thymus weight body weight ratios (T/BW) of healthy mice (Untreated) and DSS-induce colitis mice treated at 5 mg/Kg dosage.

FIG. 15 shows a replotting and reanalysis of the data presented in FIG. 11. FIG. 11 compared the data sets using a students T test. In this figure, the data sets have been compared using one-way ANOVA followed by Tukey post test. It also compares the data statistically at days 4-6, as shown in the three blown out graphs. All mice that received DSS lost weight significantly when compared to normal controls. However, when mice were treated with either Prednisolone or Mutual Prodrug 2, this weight loss was decreased as compared to vehicle only controls. There was no statistical difference between the groups that received Prednisolone or Mutual Prodrug 2.

FIG. 16 shows the replotting and reanalysis of the data presented in FIG. 12 using the statistical methods described above. All mice that received DSS displayed clinical signs of colitis as compared to the untreated mice. The severity of the disease was significantly reduced in those that received Prednisolone or Mutual Prodrug 2 as compared to those that received vehicle alone. There was no statistical difference between those groups that received Mutual Prodrug 2 or Prednisolone.

FIG. 17 is a replotting and reanalysis of the data presented in FIG. 13, using the statistical methods described above. Colon shortening is a clinical readout for severity of colitis. All mice that received DSS displayed shortening of the colon. The mice that were treated with either Mutual Prodrug 2 or Prednisolone displayed significantly less shortening than those treated with vehicle alone. There was no statistical difference between the Mutual Prodrug 2 or Prednisolone treated groups.

FIG. 18 shows a replotting and reanalysis of FIG. 14 using the statistical methods described above, with additional data showing the thymus weight:terminal body weight. In control experiments, no significant difference was observed in thymus weight versus starting body weight or versus terminal body weight in the untreated versus vehicle treated mice. As expected, the Prednisolone-treated group demonstrated a significant reduction in T/BW ratio as compared to the vehicle-treated mice. In contrast, the T/BW ratio in the Mutual Prodrug 2 treated group was not statistically different to that of the vehicle-treated group.

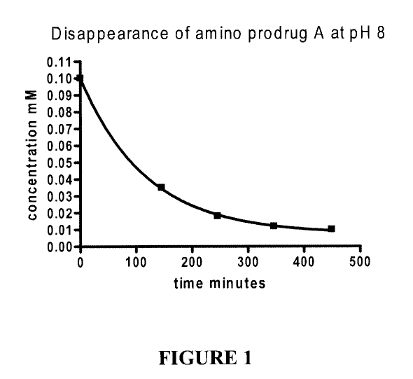

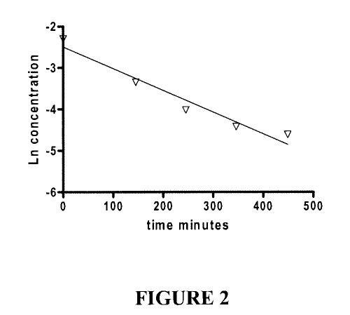

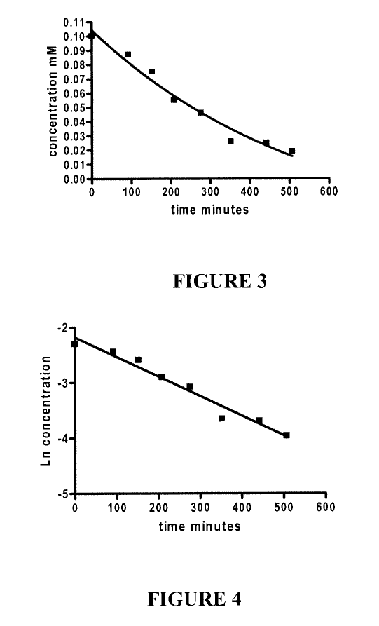

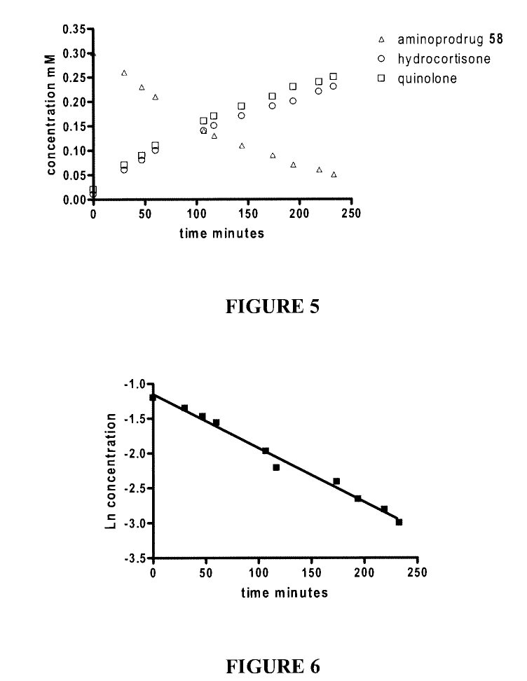

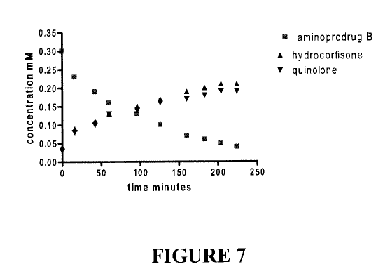

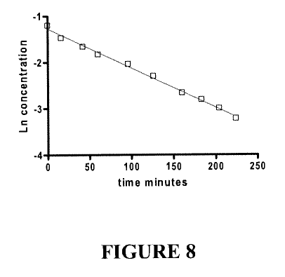

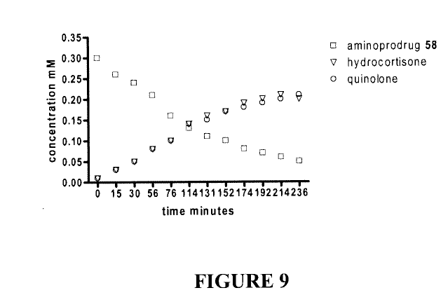

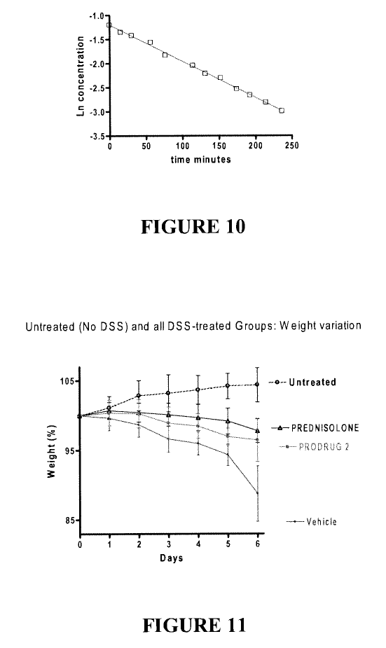

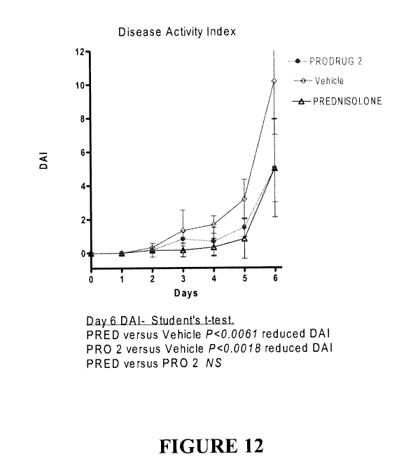

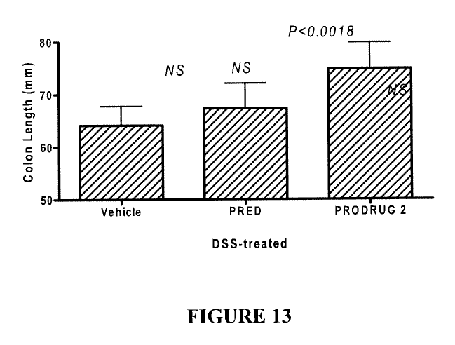

### DETAILED DESCRIPTION OF THE INVENTION METHODS

The following method describes the preparation of a series of esters of the anti-inflammatory steroids hydrocortisone, dexamethasone and budesonide at the 21-, 11- and 17-position containing azo-derivatives of the amino esters.

### Ring Closure Kinetics Compounds

In one particular example, the synthetic approach is outlined below for dexamethasone (Scheme 4). 2-Nitrocinnamic acid is reduced by catalytic reduction to the aminophenyl propionate and immediately treated with BOC anhydride. The most exposed steroidal hydroxyl group at the 21 position is selectively acylated using N,N′-dicyclohexylcarbodiimide/dimethylaminopyridine (Johnson et al., 1985). The BOC protection may be removed by bubbling anhydrous HCl through a dichloromethane (DCM) solution and the aniline 6 recovered as an HCl salt. The salt is stored at −80° C. until required. Methyl 3-(2-aminophenyl)propionate spontaneously ring closes even on storage at −20° C. (Kirby et al., 1979).

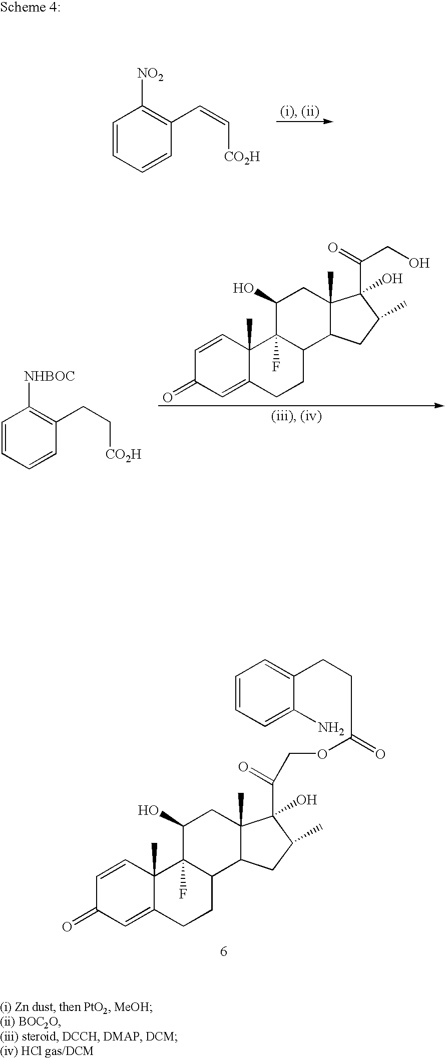

Steroidal aminophenyl acetate esters may be obtained by esterification using (o-nitrophenylacetic acid) followed by reduction of the nitro group using Zn dust in methanol and the hydrochloride salt generated as described above. Compounds suitable for evaluating mesomeric/steric effects on ring closure are obtained in a similar manner. Thus for example, the isomeric steroidal 21-methoxy-aminophenylacetates 7 and 8 may be obtained via 2-nitration of 3-methoxyphenyl acetic followed by esterification and reduction (Scheme 5). The cinnamic acid series may be obtained in a similar manner; nitration of methoxycinammic acid, reduction, BOC protection, esterification and deprotection. Methoxy groups ortho and para to the amino group may be used to discriminate between steric and electronic influences on lactamization.

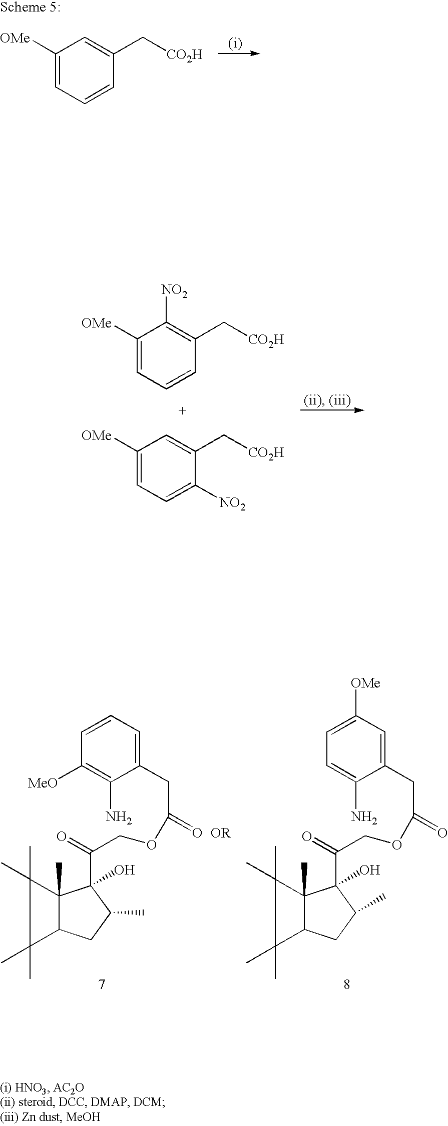

The more hindered 11-hydroxyl group may be manipulated by protecting the 17α- and 21-OH groups (Sloan et al., 1978) before acylation under more forceful conditions (or by using the phenylacetic acid chloride (Spratt et al., 1985)) followed by reduction and deprotection, to yield the 11-phenylacetate.

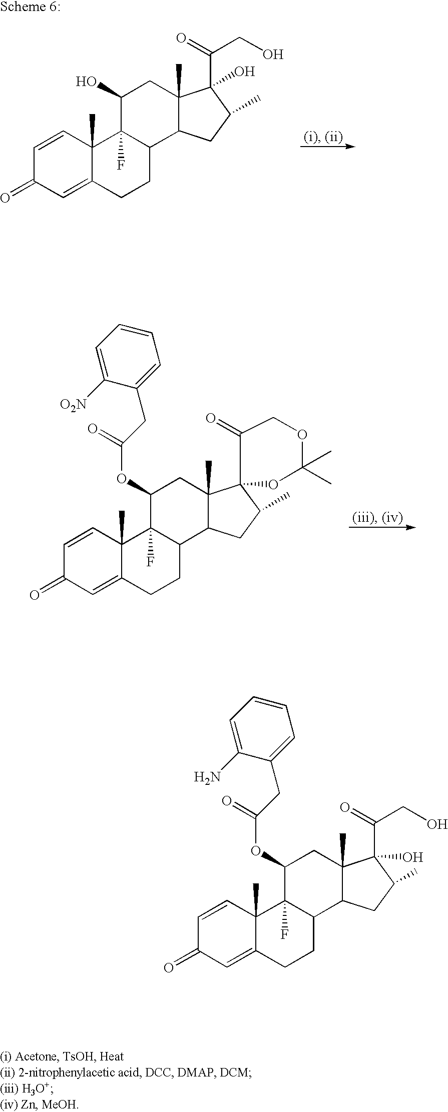

The azo prodrugs of the application may be obtained by esterification of the steroid with an azo-coupled unit. For example, the 5-ASA-steroid mutual prodrug 9, 5-ASA-dexamethasone, (Scheme 4) may be obtained by esterification of dexamethasone with compound 10 followed by deprotection of the salicylate. Similarly, a number of azo-carrier conjugates may be generated to demonstrate intestinal permeability.

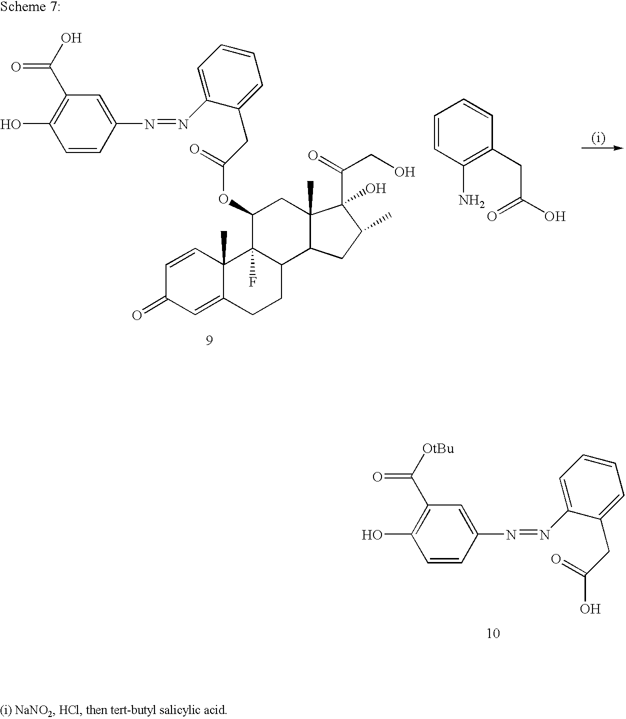

### Cyclization Kinetics

The kinetics of cyclization may be monitored using UV, HPLC or NMR as appropriate. The quinolone products of spontaneous cyclization of the steroidal propionate esters possess a λₘₐₓ at 340 nm, which allows their measurement in the presence of the ester starting material and steroidal product. The corticosteroids generally have λₘₐₓ values in the region of 240-250 nm. Alternatively, the reactions may be monitored in buffered D₂O by ¹H NMR. Although real time analysis is preferable, the reactions may also be monitored by rapid short column HPLC (RT<1 min) cooled to inhibit cyclization. In a particular aspect, the reaction may be monitored in the pH range 5-8 for both the acetate series and the propionate series, at both the 11 and 21 positions. The effect of substitution on the anilino ring may also be determined using this method to determine the optimal therapeutic activities of these compounds and their derivatives.

### Intestinal Stability

The relative stability of candidate azo-compounds may be evaluated in vitro in the presence of a range of purified pancreatic endopeptidases such as the carboxypeptidases (Gilmer et al., 2002). Compounds exhibiting maximum stability may be selected for evaluation in a rat intestinal perfusate model (Friend et al., 1985; Yano et al., 2002). Sections of rat GI tract, obtained from Bioresources, TCD (Ireland) may be gently washed and buffered at appropriate pH. The compounds may be incubated and aliquots withdrawn at intervals. Due to the complex nature of these matrices, remaining drug may be measured by reverse phase HPLC. Half-lives may be determined using Michelis Menten kinetics. Intestinal stability characteristics of selected compounds may be further probed using intestinal homogenates and human CACO-2 cell homogenates.

### Azoreduction by Colonic Bacteria In Vitro

The susceptibility of the azo-bond of candidate compounds to undergo reduction may be assessed using a simple in-vitro bacterial degradation model that employs media inoculated with commercially available Lactobacillus culture (Azad Khan et al, 1983) (this organism expresses similar azo-reductases to colonic bacteria). The medium may be supplemented with the coenzymes NADPH and FAD. The reduction may be further investigated using rat or guinea pig colonic perfusate that has been saturated with CO₂ (see for example Yano, 2002).

### Anti-Inflammatory Activity

The efficacy of the prodrugs in vivo may be assessed in an animal model. In acute murine dextran sodium sulphate-induced (DSS) colitis, NFκB DNA-binding activity is increased and the inflammatory pathology observed parallels that seen in human inflammatory bowel disease/colitis (Okayasu et al., 1990).

The balb/c mouse is a suitable strain to use for this experiment, as it is known to be susceptible to oral DSS, resulting in colitis (Egger et al., 2000). Female mice 6-8 weeks of age may be used to ensure the animals are mature and comparable in weight. Based on the Resource Equation Method, where E (error degrees of freedom)=N (number of experimental units)−T (number of treatments), and with a total of 21 groups, 3 animals per group is considered a suitable sample size (E=42) (Festing et al., 2002). Twelve groups of mice may be administered 2.5% DSS in distilled water ad libitum for a period of 7 days. Previous experiments have shown this concentration of DSS to be sufficient to induce mild colitis, with low levels of weight loss or grade III lesions, which are representative of extensive mucosal destruction (Egger et al., 2000). The remaining 9 control groups may receive distilled water only. Mice may then be treated with pro-drug by oral administration. Eight of the groups that have been given DSS will receive 2 different amounts of pro-drug twice daily for 1-4 consecutive days. Mice may be sacrificed by cervical dislocation 1 h after receiving the last dose. As a control for the mice recovering naturally from the induced colitis, a DSS-treated group may be sacrificed on each of the four days at the same time the pro-drug-treated mice are sacrificed. The control mice, who will have received water only, may be given the same doses of pro-drug as the DSS-treated animals. One control group may be left untreated.

The intestines may be removed for histological examination and RNA extraction, therefore a further 3 untreated mice are required to practice dissection, histology and RNA extraction techniques. A section of the colon may be fixed in 10% zinc-formalin and embedded in paraffin. A transverse section may be stained routinely with haematoxylin and eosin. The severity of mucosal injury is graded on a scale of 0-III: grade 0: normal; grade I: distortion and/or destruction of the bottom third of the glands; grade II: erosions/destruction of bottom two-thirds or loss of all glands with remaining surface epithelium; grade III, loss of all glands (Egger et al., 2000; Festing et al., 2002; Egger et al., 1997). Reverse transcriptase polymerase chain reaction may be carried out on extracted RNA to determine levels of NFκB-dependent pro-inflammatory cytokines IL-1 and TNF-α, which are up-regulated in DSS-induced colitis (Egger et al., 2000). Depending on the results obtained from these studies, the experiments may be repeated with different concentrations of pro-drug and different numbers of doses if necessary.

### GI Permeability Assessment

The prodrugs have been designed to be excluded from the absorption process, thus permitting their transit to the colon. Compounds for further evaluation may be selected on the basis of their poor permeability characteristics. The GI behaviour of the compounds may be evaluated first using the everted intestinal sac (Wilson and Wiseman, 1954), which permits direct evaluation and comparison of drug transfer across the in vitro barrier and the intestinal perfusion model described below.

### In Vivo Model—Single Pass Perfusion: Intestinal Perfusion

This is essentially an isolated gut loop model in which depletion of drug from the lumen is monitored (Komiya et al., 1980). The gut remains in situ in an anaesthetized animal so that normal function is perturbed as little as possible. There are limitations to the model in that splancnic blood flow is naturally variable and is decreased under anesthesia. A midline incision of the animal allows gentle exposure of a 20-40 cm midileal portion of the intestine. This portion is selected because of its accessibility and suitable vasculature to facilitate cannulation. Mesenteric arcades to adjacent portions are tied off. The intestine is cut and cannulated with Tygon tubing for perfusion. The mesenteric vein is cannulated with an appropriate size of Silastic or polyethylene tubing and venous blood is collected in heparinized (or equivalent), calibrated centrifuge tubes. After gentle washing of the intestinal lumen with isotonic phosphate buffer at 37° C., drug solution is perfused into the proximal section of the segment allowed to flow through the segment at 0.2 ml/min and collected at the distal end of the lumenal segment. The isolated intestine is kept warm and moist by frequent application of warm (37° C.) saline to a gauze pad covering the intestine, which is covered by a dental rubber dam. A small lamp is also used to maintain the preparation at 37° C. The drug is perfused through the intestine for up to 120 minutes with perfusate collection at regular intervals. Perfusate is assayed and drug disappearance from perfusion fluid is monitored over the time course of the experiment.

### EMBODIMENTS AND ASPECTS OF THE PRESENT APPLICATION

In one particular aspect as described above, the experiments provide an evaluation of the methods as a means of targeting the colon with anti-inflammatory steroids including novel mutual prodrugs of the two principal therapies used in IBD.

In another aspect of the application, there is provided a method for the synthesis of several new families of novel steroidal esters bearing anilines and azo carriers. The method provides comprehensive study of the cyclization kinetics of aminophenyl acetate and propionate esters under simulated physiological conditions and insight into the electronic and steric ring substitution effects on closure kinetics. In addition, the method provides a determination of the aqueous, enzymatic and intestinal stability of 21 and 11-corticosteroid esters. In yet another aspect, the method allows the determination of the rate of reduction of steroid aza conjugates and dependence on steroid and ring substitution effects.

In one embodiment, there is provided a compound of the formula Ia:

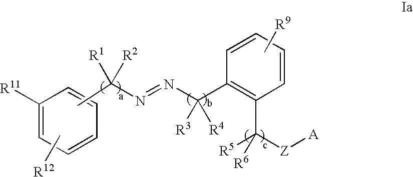

wherein: A is hydrogen or is a residue of a hydroxyl-bearing drug; Z is selected from the group consisting of —C(O)—, —S(O)—, —OC(O)—, —OC(O)NR¹³—, —S—C(O)—, —SC(O)NR¹³—, —C(O)NR¹³—, —NR¹³C(O)NR¹³—, —OS(O)—, —OS(O)₂—, —S(O)₂NH— and —OPO(OH)—; each R¹, R², R⁵ and R⁶ is independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted; each R³ and R⁴ is independently selected from the group consisting of hydrogen (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each substituted or unsubstituted; R¹¹ is selected from the group consisting of hydrogen, hydroxy, (C₁₋₃)alkoxy, and —C(O)OR¹⁰; each R⁹ and R¹² is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, —SO₃R¹³, P₃R¹³, (C₁₋₃)alkoxy, each alkyl, cycloalkyl, aryl and heteroaryl substituted or unsubstituted; or R¹¹ and R¹² when substituted adjacent in the phenyl ring, are taken together form an optionally substituted heterocyclic ring; R¹⁰ is hydrogen or (C₁₋₃)alkyl; each R¹³ is independently hydrogen or (C₁₋₃)alkyl, and each a, b and c is independently 0, 1 or 2; or a pharmaceutically acceptable salt thereof, optionally in the form of a single stereoisomer or mixture of stereoisomers. In one variation, R¹¹ and R¹² are taken together to form an optionally substituted heterocyclic ring. In another variation, R¹¹ and R¹² are taken together to form acetonidyl-4-one. In yet another variation, R¹¹ is C(O)O—R¹⁰, and each of R¹² and R¹⁰ is hydrogen.

In another embodiment, there is provided a compound of the formula I:

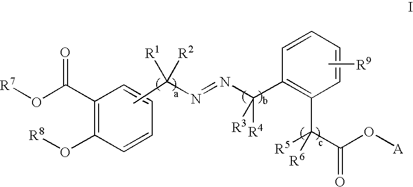

wherein: A is hydrogen or is a residue of a hydroxyl-bearing drug; each R¹, R², R⁵ and R⁶ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted; each R³ and R⁴ are independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each substituted or unsubstituted; R⁷ is hydrogen or is (C₁₋₃)alkyl; R⁸ is hydrogen or is (C₁₋₃)alkyl; R⁹ is selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, each substituted or unsubstituted; and each a, b and c is independently 0, 1 or 2; or a pharmaceutically acceptable salt thereof, optionally in the form of a single stereoisomer or mixture of stereoisomers. In one variation, R⁷ and R⁸ are hydrogen, and c is 1 or 2. In another variation, a and b are both 0, and c is 1 or 2. In yet another variation, R⁹ is hydrogen; in another variation, R⁷ and R⁸ are hydrogen, R⁹ is hydrogen, and a and b are both 0.

In another embodiment, there is provided a compound of the formula II:

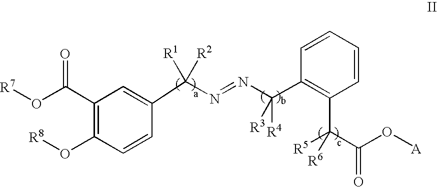

wherein: A is hydrogen or is a residue of a hydroxyl-bearing drug; each R¹, R², R⁵ and R⁶ are independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted; each R³ and R⁴ are independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each substituted or unsubstituted; R⁷ is hydrogen or is (C₁₋₃)alkyl; R⁸ is hydrogen or is (C₁₋₃)alkyl; each a, b and c is independently 0, 1 or 2; or a pharmaceutically acceptable salt thereof, optionally in the form of a single stereoisomer or mixture of stereoisomers. In one variation, R⁷ and R⁸ are hydrogen, and c is 1 or 2; in another embodiment, a and b are both 0, and c is 1 or 2. In yet another variation, R⁷ and R⁸ are hydrogen, and a and b are both 0.

In yet another embodiment, there is provided a compound of the formula III:

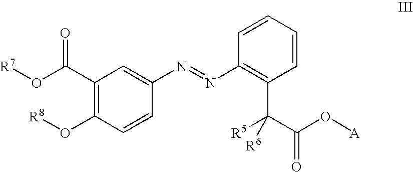

wherein: A is hydrogen or is a residue of a hydroxyl-bearing drug; R⁵ and R⁶ are independently hydrogen or are independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted; R⁷ is hydrogen or is (C₁₋₃)alkyl; R⁸ is hydrogen or is (C₁₋₃)alkyl; or a pharmaceutically acceptable salt thereof, optionally in the form of a single stereoisomer or mixture of stereoisomers. In one variation, R⁵ and R⁶ are hydrogen; in another embodiment, R⁵, R⁶, R⁷ and R⁸ are each hydrogen. In another variation, R⁷ and R⁸ are each (C₁₋₃)alkyl.

In one aspect of the present application for each of the above embodiments, variations and aspects, A or the hydroxyl-bearing drug of any of the disclosed compounds is selected from the group consisting of an anti-inflammatory drug, an anti-cancer drug, an imaging agent; a vaccine, an antigen, an anti-infective drug, a peptide, an antisense molecule and a protein. In one variation, the hydroxyl-bearing drug is an anti-inflammatory drug. In another variation, the anti-inflammatory drug is a steroid. In yet another variation, the steroid is selected from the group consisting of hydrocortisone, dexamethasone, budesonide esterified at the 21-, 11- and 17-positions, respectively and prednisolone.

In one variation of each of the above embodiment, aspects and variations thereof, R¹¹ and the linker comprising the azo group (i.e., —N═N—) is 1,4-di-substituted (i.e., para) on the phenyl ring. In another variation, R¹² is H. In yet another variation of the above, a is 0; or a and b are both 0. In another variation of the above, R⁹ is H.

In another aspect of the present application, A or the hydroxyl-bearing drug of any of the disclosed compounds is selected from the group consisting of nitroimidazoles, quinolines such as nalidixic acid, fluoroquinolones, such as ciprofloxacin or levofloxacin, aminoglycosides, such as amikacin, gentamicin, kanamycin, neomycin, netilmicin, paromomycin, streptomycin, tobramycin and apramycin, leucovorin, topotecan, irinotecan, methotrexate, bevacizumab, cetuximab, panitumumab and infliximab. In certain variation of the above, the drug is an amine bearing drug (i.e., a drug with an amine group) such as celecoxib, or a thiol bearing drug.

In one aspect, the compound of present application is selected from the group consisting of:

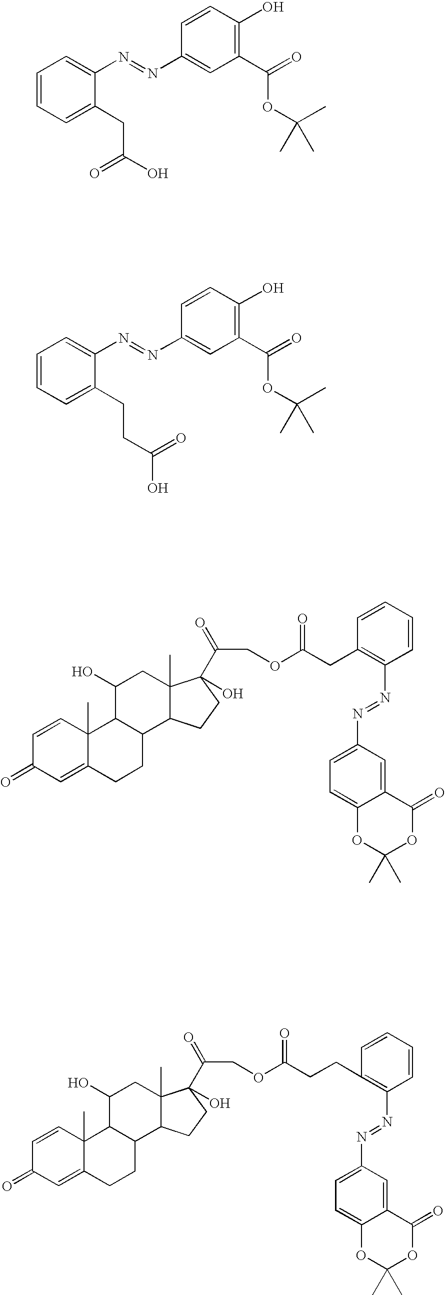

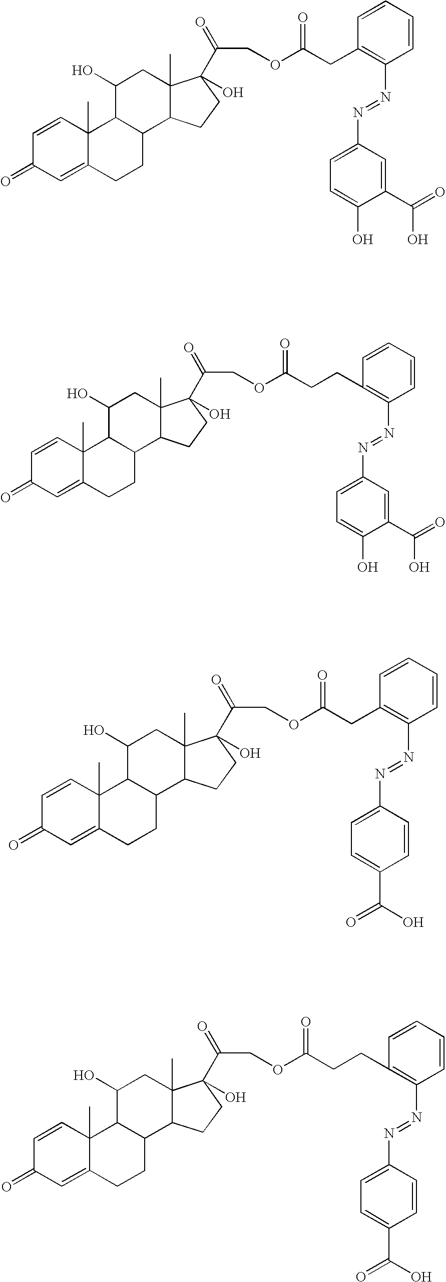

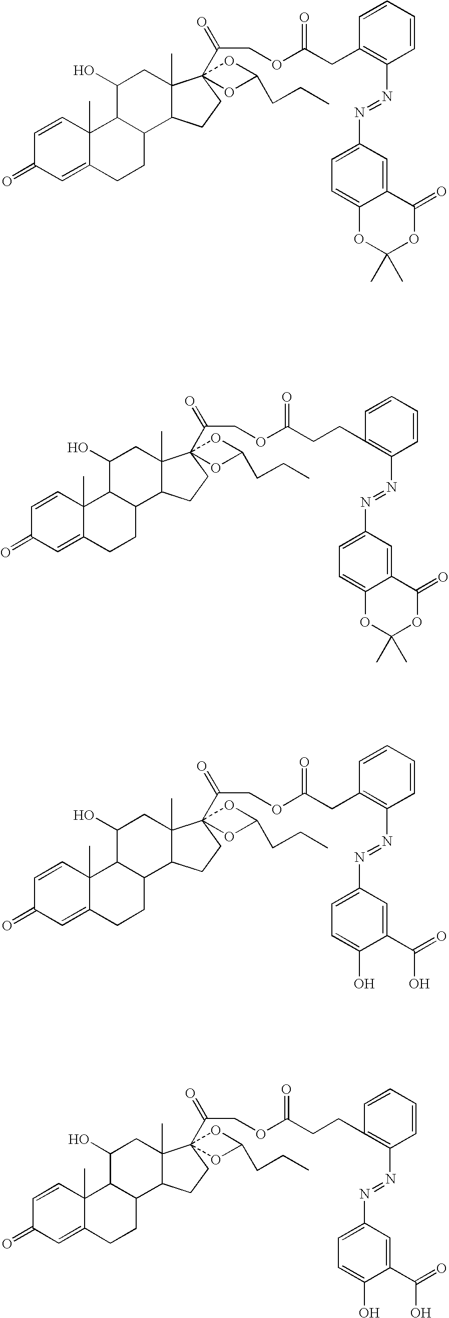

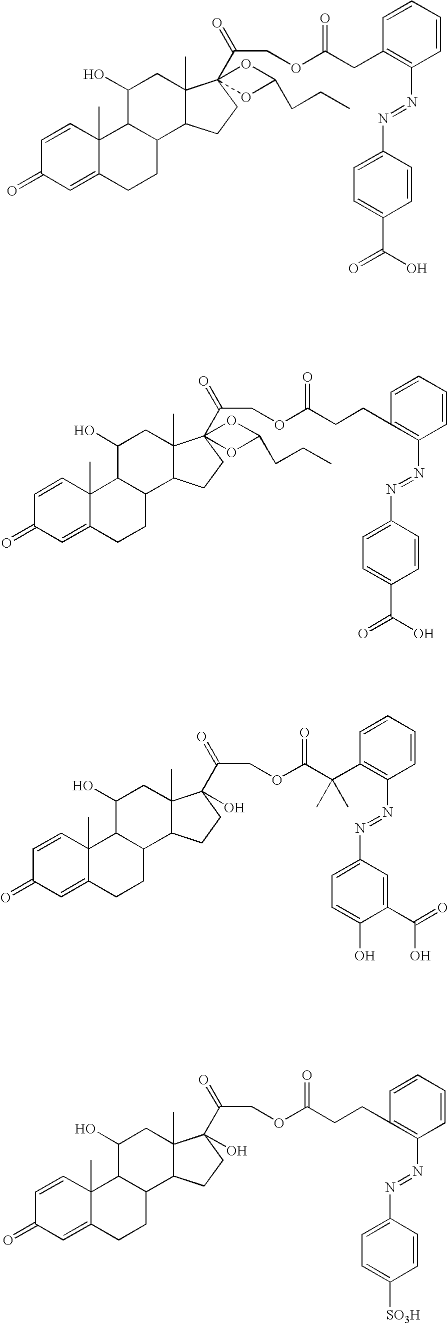

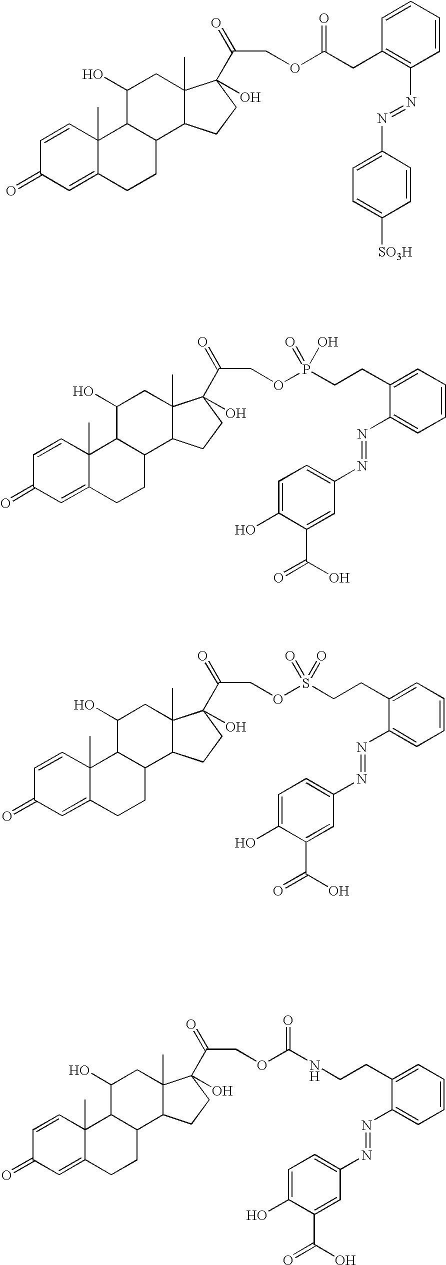

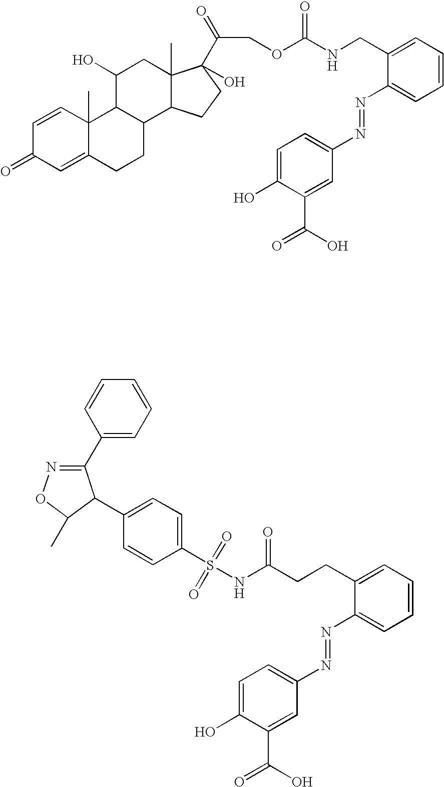

In one aspect, there is provided a pharmaceutical composition comprising a therapeutically effective amount of any of the disclosed compounds provided herein and a pharmaceutically acceptable excipient.

In one embodiment, there is provided a method for decreasing NFκB DNA-binding activity in a patient, the method comprising administering a therapeutically effective amount of any compound or composition of the present application to the patient. In one variation, the therapeutically effective amount is effective to reduce, alleviate or treat inflammatory bowel disease (IBD). In another variation, the therapeutically effective amount is effective to reduce, alleviate or treat ulcerative colitis or Crohn's disease.

In one aspect, there is provided a method of treating inflammatory bowel disease (IBD), comprising administering a therapeutically effective amount of any compound or composition of the present application to a mammal in need of such treatment. In another aspect, there is provided a method of treating Crohn's disease or ulcerative colitis, comprising administering a therapeutically effective amount of any compound or composition of the present application to a mammal in need of such treatment. In one variation of the above, the amount of compound or composition administered is enough to maintain remission. In another variation of the above method, 5ASA-5ASA is administered.

In another aspect, there is provided a method of reducing, alleviating or treating acute ulcerative colitis comprising administering a therapeutically effective amount of 5ASA-steroid, wherein steroid is hydrocortisone, dexamethasone, budesonide esterified at the 21-, 11- and 17-positions, respectively, or prednisolone. In one variation, the steroid is prednisolone.

In another embodiment, there is provided a method of treating Collagenous colitis, Lymphocytic colitis, Ischaemic colitis, Diversion colitis, Behçet's syndrome, Infective colitis, or Indeterminate colitis comprising administering a therapeutically effective amount of any compound or composition of the present application to a mammal in need of such treatment. In another aspect there is provided a method of treating Amebiasis, Clostridium Difficile Infection, Pseudomembranous colitis, Diverticulitis, Gastroenteritis, Gastrointestinal Cancers, or Irritable Bowel Syndrome (IBS) comprising administering a therapeutically effective amount of any compound or composition of the present application to a mammal in need of such treatment.

In another embodiment, there is provided a method for treating or alleviating an inflammatory condition in a mammal, the method comprising delivering an effective amount of a COX2 inhibitor to the colon, wherein the COX2 inhibitor is the residue of a hydroxyl bearing drug of any of the compound or compositions as provided herein. In another embodiment, there is provided a method for treating gastrointestinal cancer in a mammal, the method comprising delivering an effective amount of a COX2 inhibitor to the colon, wherein the COX2 inhibitor is the residue of a hydroxyl bearing drug of any of the compound or compositions as provided herein.

Also included in the above embodiments, aspects and variations are salts of amino acids such as arginate and the like, gluconate, and galacturonate. Some of the compounds of the present application may form inner salts or Zwitterions. Some of the compounds of the present application can exist in unsolvated forms as well as solvated forms, including hydrated forms, and are intended to be within the scope of the present application. Also provided are pharmaceutical compositions comprising pharmaceutically acceptable excipients and a therapeutically effective amount of at least one compound of this application.

Pharmaceutical compositions of the compounds of this application, or derivatives thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, 5% dextrose in water or buffered sodium or ammonium acetate solution. Such formulations are especially suitable for parenteral administration but may also be used for oral administration. Excipients, such as polyvinylpyrrolidinone, gelatin, hydroxycellulose, acacia, polyethylene glycol, mannitol, sodium chloride, or sodium citrate, may also be added. Alternatively, these compounds may be encapsulated, tableted, or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols or water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax. The amount of solid carrier varies but, preferably, may be between about 20 mg to about 1 g per dosage unit. The pharmaceutical preparations are made following the conventional techniques of pharmacy involving milling, mixing, granulation, and compressing, when necessary, for tablet forms; or milling, mixing, and filling for hard gelatin capsule forms. When a liquid carrier is used, the preparation may be in the form of a syrup, elixir, emulsion, or an aqueous or non-aqueous suspension. Such a liquid formulation may be administered directly p.o. or filled into a soft gelatin capsule. Suitable formulations for each of these methods of administration may be found in, for example, Remington: The Science and Practice of Pharmacy, A. Gennaro, ed., 20th edition, Lippincott, Williams & Wilkins, Philadelphia, Pa.

In one variation, there is provided the above compound, or a pharmaceutically acceptable salt thereof, optionally in the form of a single stereoisomer or mixture of stereoisomers thereof.

### DEFINITIONS

Unless specifically noted otherwise herein, the definitions of the terms used are standard definitions used in the art of organic synthesis and pharmaceutical sciences.

An “alkyl” group is a straight, branched, saturated or unsaturated, aliphatic group having a chain of carbon atoms, optionally with oxygen, nitrogen or sulfur atoms inserted between the carbon atoms in the chain or as indicated. A (C₁-C₁₀)alkyl, for example, includes alkyl groups that have a chain of between 1 and 10 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, and the like. An alkyl group may also be represented, for example, as a —(CR¹R²)ₐ— group where R¹ and R² are independently hydrogen or are independently substituted as provided herein; and for example, a is 0, 1 or 2.

An alkyl as noted with another group such as an aryl group, represented as “arylalkyl” for example, is intended to be a straight, branched, saturated or unsaturated aliphatic divalent group with the number of atoms indicated in the alkyl group (as in (C₁-C₁₀)alkyl, for example) and/or aryl group or when no atoms are indicated means a bond between the aryl and the alkyl group. Nonexclusive examples of such group include benzyl, phenylethyl and the like.

An “alkylene” group is a straight, branched, saturated or unsaturated aliphatic divalent group with the number of atoms indicated in the alkyl group; for example, a —(C₁-C₃)alkylene- or —(C₁-C₃)alkylenyl-.

An “aryl” group is a monocyclic or bicyclic aromatic hydrocarbon group having 5 to 8 atoms in the ring, such as a phenyl. The monocyclic aryl groups are typically are 5 to 7 membered rings, and the bicyclic aryl groups are typically 7 to 8 membered rings.

The term “heteroaryl,” as used herein, means an aryl group containing from, for example, about 3 to about 30 atoms, preferably from about 6 to about 18 atoms, more preferably from about 6 to about 14 atoms, and most preferably from about 6 to about 10 atoms and from 1 to 3 heteroatoms (e.g., N, O or S). Examples of such groups include pyrrolyl, imidazolyl, pyrazolyl, furanyl, oxazolyl, isooxazolyl, thiofuranyl, thiazolyl, isothiazolyl, indolyl, isoindolyl, benzofuranyl, quinolinyl, pyridinyl, pyridazinyl, pyrazinyl, triazolyl and benzotriazolyl.

A “cyclyl” such as a monocyclyl or polycyclyl group includes monocyclic, or linearly fused, angularly fused or bridged polycycloalkyl, or combinations thereof. Such cyclyl group is intended to include the heterocyclyl analogs. A cyclyl group may be saturated, partially saturated or aromatic.

“Halogen” or “halo” means fluorine, chlorine, bromine or iodine.

A “heterocyclyl” or “heterocycle” is a cycloalkyl group wherein one or more of the atoms forming the ring is a heteroatom that is a N, O or S. The cycloalkyl may be saturated, partially saturated or aromatic. Non-exclusive examples of heterocyclyl include piperidyl, 4-morpholyl, 4-piperazinyl, pyrrolidinyl, 1,4-diazaperhydroepinyl, acetonidyl-4-one, 1,3-dioxanyl, thiophenyl, furanyl, pyrrolyl, pyrazolyl, pyridinyl, pyrimidinyl pyridazinyl, pyranyl and the like.

The term “alkoxy” includes linear or branched alkyl groups that are attached to divalent oxygen. The alkyl group is as defined above. Examples of such substituents include methoxy, ethoxy, t-butoxy, and the like. The term “alkoxyalkyl” refers to an alkyl group that is substituted with one or more alkoxy groups. The term “heteroaryloxy” refers to a heteroaryl group that is substituted with one or more alkoxy groups. The term “aryloxy” refers to an aryl group that is attached to an oxygen, such as phenyl-O—, etc.

As used herein, where a divalent group is represented by a group -Z- as described herein, or generically as -A-B-, as shown below for example, it is intended to also represent a group that may be attached in both possible permutations, as noted in the two structures below.
[symbol] -A-B- [symbol] may also be [symbol] -B-A-

[symbol]

For example, when a divalent group such as the group “—NR¹³C(O)—” is provided, for example, the group is intended to also include both the divalent group —NR¹³C(O)— and also the divalent group —C(O)NR¹³—.

A “hydroxyl-bearing drug” means a drug or a biologically active compound that is functionalized or that is substituted with a hydroxyl (i.e., —OH) group. Accordingly, the residue of a hydroxyl-bearing drug is the component of the drug or the biologically active compound without the hydroxyl group. Drugs employed in the present application may include an anti-inflammatory drug, an anti-cancer drug, an imaging agent, particularly as used in the imaging of colon diseases; a vaccine, an antigen, an anti-infective drug, a peptide, an antisense molecule, and a protein. Such drugs can include, but are not limited to steroidal compounds (e.g. esters of hydrocortisone, dexamethasone or budesonide), nitroimidazoles (e.g. metronidazole), antibiotics, (e.g. quinolines such as nalidixic acid, fluoroquinolones, such as ciprofloxacin and levofloxacin), chemotherapeutics (e.g. leucovorin, topotecan), and antibodies, (e.g. bevacizumab, cetuximab and panitumumab).

“Pharmaceutically acceptable salts” means salt compositions that is generally considered to have the desired pharmacological activity, is considered to be safe, non-toxic and is acceptable for veterinary and human pharmaceutical applications. Such salts include acid addition salts formed with inorganic acids such as hydrochloric acid, hydrobromic acid, sulfuric acid, phosphoric acid, and the like; or with organic acids such as acetic acid, propionic acid, hexanoic acid, malonic acid, succinic acid, malic acid, citric acid, gluconic acid, salicylic acid and the like.

“Pro-drug” or “prodrug” as used herein, means a bioprecursor or pharmaceutically acceptable compound that may be convertible or degradable in the body, specifically in the colon, to produce a biologically active compound(s) of the invention (for example, the intermediate aniline or the lactam and the active drug). In particular, the compounds of the present application may be reduced by an in vivo azoreductase such as microflora azoreductase.

“Therapeutically effective amount” means a drug amount that elicits any of the biological effects listed in the specification. A therapeutically effective amount also means an amount of the composition comprising a compound/agent or pro-drug, as disclosed herein, that is useful or capable of supporting an observable change in the level of one or more biological activity characteristic of a compound/agent or pro-drug, or a dosage amount that is sufficient to impart a beneficial effect. Such beneficial effect may comprise an amelioration of a symptom for the recipient or patient. In certain aspects, the therapeutically effective amount may be related to the healing or curing of disease. As is known in the art, a number of considerations may be taken into account to determine the therapeutically effective amount. Examples of such considerations may be found in, for example, in Gilman, A. G., et al, Goodman and Gilman's The Pharmacological Basis of Therapeutics, 8^{th} ed., McGraw-Hill (1990), and Remington's Pharmaceutical Sciences, 17^{th} ed., Mack Publishing Co., Easton, Pa. (1990).

“Substituted or unsubstituted” or “optionally substituted” means that a group such as, for example, alkyl, aryl, heterocyclyl, (C₁-C₈)cycloalkyl, heterocyclyl(C₁-C₈)alkyl, aryl(C₁-C₈)alkyl, heteroaryl, heteroaryl(C₁-C₈)alkyl, and the like, unless specifically noted otherwise, may be unsubstituted or, may substituted by 1, 2 or 3 substituents selected from the group such as halo, nitro, trifluoromethyl (—CF₃), trifluoromethoxy, methoxy, carboxy, —NH₂, —OH, —SH, —SCH₃, —NHCH₃, —N(CH₃)₂, —SMe, cyano and the like.

### Experimental

The following procedures may be employed for the preparation of exemplary compounds of the present application. The starting materials and reagents used in preparing these compounds are either available from commercial suppliers such as the Sigma Aldrich Chemical Company (Milwaukee, Wis.), Bachem (Torrance, Calif.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In some cases, protective groups may be introduced and finally removed. Suitable protective groups for amino, hydroxy, and carboxy groups are described in Greene et al., Protective Groups in Organic Synthesis, Second Edition, John Wiley and Sons, New York, 1991. Standard organic chemical reactions can be achieved by using a number of different reagents, for examples, as described in Larock: Comprehensive Organic Transformations, VCH Publishers, New York, 1989.

In one variation, the compounds of this application can be synthesized by the steps outlined in Schemes 8-11 below. The schemes demonstrate the preparation of different homologs of the pro-drugs.

### General Experimental Procedures

Uncorrected melting points were obtained using a Stuart® melting point SMP11 melting point apparatus. Infra-red (IR) spectra were obtained using a Perking Elmer 205 FT Infrared Paragon 1000 spectrometer. Band positions are given in cm⁻¹. Solid samples were obtained by KBr disk; oils were analyzed as neat films on NaCl plates. UV spectroscopy was performed on a Cary 3E UV-VIS spectrophotometer. ¹H and ¹³C spectra were recorded at 27° C. on a Bruker DPX 400 MHz FT NMR spectrometer (400.13 MHz ¹H, 100.16 MHz ¹³C), in either CDCl₃ or CD₃OD, (tetramethylsilane as internal standard). For CDCl₃, ¹H NMR spectra were assigned relative to the TMS peak at 0.00 δ and ¹³C NMR spectra were assigned relative to the middle CDCl₃ triplet at 77.00 ppm. For CD₃OD, 1H and ¹³C spectra were assigned relative to the centre peaks of the CD₃OD multiplets at 3.30 δ and 49.00 ppm respectively. Coupling constants were reported in hertz (Hz). For ¹H NMR assignments, chemical shifts are reported: shift values (number of protons, description of absorption (s=singlet, d=doublet, t=triplet, q=quartet, m=multiplet), coupling constant(s) where applicable, proton assignment). LRMS was performed on a micromass mass spectrophotometer (El mode) at the Department of Chemistry, Trinity College. Flash chromatography was performed on Merck Kieselgel 60 particle size 0.040-0.063 mm thin layer chromatography (TLC) for which Rf values are quoted, was performed on silica gel Merck F-254 plates. Compounds were visually detected by absorbance at 254 nm+/or vanillin staining.

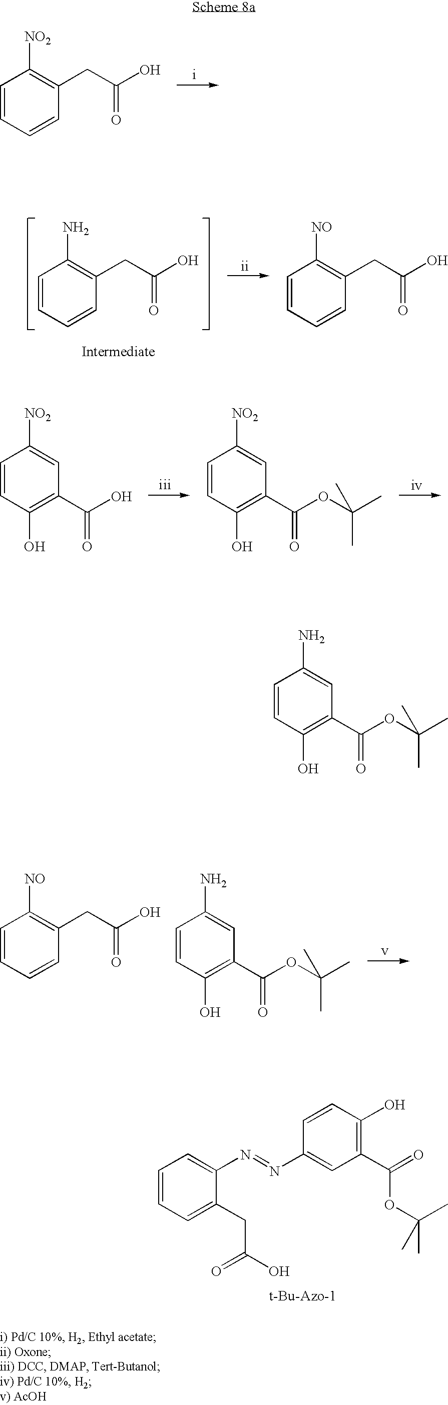

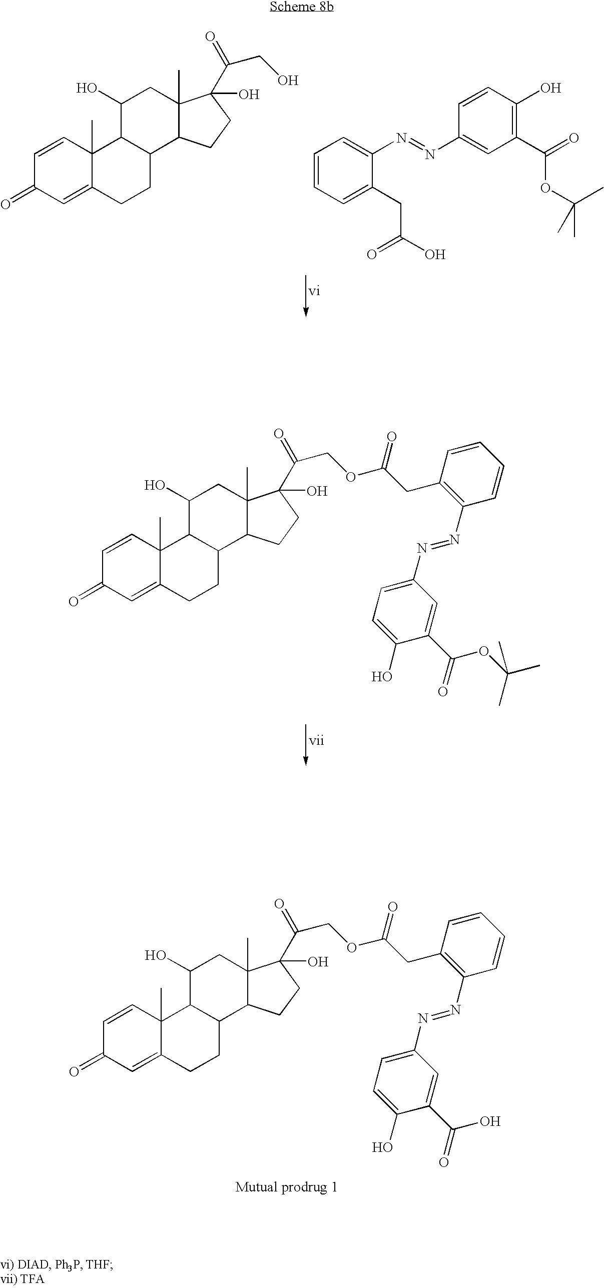

### 2-Nitrosophenylacetic acid

To a solution of 2-nitrophenylacetic acid (5 g, 0.0276 mol) in ethyl acetate 100 mL, palladium 10% on charcoal was added on catalyst amount (5%, 0.25 g). The reaction mixture was stirred under hydrogen atmosphere at room temperature during two hours, after that time, TLC analysis showed reaction completion (ethyl acetate-dichloromethane 40:60). Reaction mixture was filtered over filter agent to remove the Palladium 10% on charcoal and solvents were removed under reduced pressure. The crude 2-aminophenylpropionic was dissolved in distillated water 50 mL and dichloromethane 50 mL. Oxone® 2 equivalents (33.9 g, 0.0552 mol) were dissolved in distillated water 50 mL and added to the reaction mixture. At this point the reaction pH=4 so the 2-nitrosophenylacetic acid is being transferred to the organic layer as soon as it is formed. After 2 hours reaction mixture is transferred to a separate funnel. The organic layer was collected and aqueous phase was washed 2×25 mL dichloromethane. The organic phase is collected and dry with Na₂SO₄. Solvents were removed under reduced pressure to afford 4.21 g of product as dark oil. The crude mixture was flash columned using dichloromethane:ethyl acetate (70:30) to yield 3.48 g product as brown crystals (70%): IRᵥₘₐₓ (KBr): 1691.86 (C═O) cm⁻¹. ¹H NMR δ (CDCl₃): 9.00 (1H, s, COOH), 7.24 (2H, t, J 8.54 and 7.53 Hz), 7.04 (1H, t, J 7.53 and 7.53 Hz), 6.92 (1H, d, J=7.53 Hz), 3.57 (2H, s). ¹³C NMR δ (CDCl₃): 178.02 (C, C-8, C═O), 141.86 (C, C-1), 127.50 (CH, C-4), 124.77 (C, C-2), 124.15 (CH, C-3), 122.05 (CH, C-5), 109.50 (CH, C-6), 35.84 (CH₂, C-7).

### 5-(2-Carboxymethyl-phenylazo)-2-hydroxybenzoic acid tert-butyl ester

To a solution of 2-nitrosophenylacetic acid (1.0 g, 0.0061 mol) in glacial acetic acid (20 mL), another solution of 5-aminosalicylic tert-butyl ester (1.26 g, 0.0061 mol) in glacial acetic acid (20 mL) was added dropwise over 15 minutes. Reaction was left stirring 48 hours until completion by TLC dichloromethane:ethyl acetate (50:50). Solvents were removed under reduced pressure to afford the product as black oil. This was flash columned using dichloromethane as mobile phase to yield 1.49 g of the product as orange crystals (69%): mp: 108-110° C. IRᵥₘₐₓ (KBr): 3434.51 cm⁻¹, 1714.14 cm⁻¹, 1670.39 cm⁻¹, 1587.05 cm⁻¹. ¹H NMR δ (CDCl₃): 11.49 (1H, s), 8.37 (1H, d, J=2.48 Hz), 7.95 (1H, dd, J 9 and 2.48 Hz), 7.76 (1H, d, J=8.04 Hz), 7.40 (3H, m), 6.97 (1H, d, J=9.04 Hz), 4.10 (2H, s), 1.64 (9H, s). ¹³C NMR δ (CDCl₃): 177.05 (C, C-8, C═O), 169.14 (C, C-15, C═O), 163.83 (C, C-12), 149.40 (C, C-2), 144.84 (C, C-9), 133.26 (CH, C-1), 131.01 (CH, C-5), 130.49 (CH, C-6), 127.82 (CH, C-10), 127.62 (CH, C-4), 127.26 (CH, C-14), 117.99 (CH, C-13), 115.53 (CH, C-3), 113.32 (C, C-11), 83.24 (C, C-16), 37.40 (CH₂, C-7), 27.69 (3×CH₃, C-17).

### Tert-Butyl Ester Protected Mutual Prodrug 1

To a solution of 5-(2-carboxymethyl-phenylazo)-2-hydroxybenzoic acid tert-butyl ester (0.40 g, 0.0011 mol), prednisolone (1.1 eq, 1.18 g, 0.0013 mol) and triphenylphosphine (3 eq, 0.88 g, 0.0033 mol) in dry THF (50 mL), diisopropylazodicarboxylate 95% (DIAD) was added (3 eq, 0.66 mL, 0.0033 mol) dropwise over 12 minutes after the reaction temperature reach 40° C. Reaction was stirring at 40° C. during an hour and reaction was left overnight at room temperature under nitrogen atmosphere. TLC analysis (dichloromethane) shows completion. Solvents were removed under reduced pressure to afford the product as orange oil. This was flash columned using dichloromethane:ethyl acetate (50:50), after this column the triphenylphosphine oxide was removed and a second flash column is needed hexane (200 mL), hexane:ethyl acetate (70:30) to yield the product 0.33 g as orange crystals (43%): mp: 124-126° C. IRᵥₘₐₓ (KBr): 3436.29 cm⁻¹, 1726.17 cm⁻¹, 1659.61 cm⁻¹, 1616.09 cm¹. ¹H NMR δ (CDCl₃): 11.49 (1H, s), 8.44 (1H, s), 8.05 (1H, d, J=9 Hz), 7.75 (1H, d, J=7.52 Hz), 7.40 (3H, m), 7.24 (1H, d, J 10.04), 7.06 (1H, d, J=9 Hz), 6.25 (1H, s), 5.98 (1H, s), 4.27 (2H, s), 2.68-0.84 (prednisolone envelope, 19H), 1.67 (9H, s). ¹³C NMR δ (CDCl₃): 204.40 (C, C-20, C═O), 186.26 (C, C-3, C═O), 171.36 (C, C-22, C═O), 170.10 (C, C-5), 169.09 (C, C-13, C═O), 163.85 (C, C-10′), 156.25 (CH, C-1), 149.53 (C, C-2′), 145.07 (C, C-7′), 133.25 (C, C-1′), 130.98 (CH, C-5′), 130.53 (CH, C-6′), 129.89 (CH, C-8′), 127.85 (CH, C-2), 127.17 (CH, C-4′), 125.52 (CH, C-12′), 121.79 (CH, C-4), 118.14 (CH, C-11′), 115.14 (CH, C-3′), 113.26 (C, C-9′), 89.17 (C, C-17), 83.30 (C, C-14′), 69.51 (CH, C-11), 67.98 (CH₂, C-21), 54.86 (CH, C-9), 50.86 (CH, C-14), 47.26 (C, C-13), 43.68 (C, C-10), 38.85 (CH₂, C-12), 36.44 (CH₂, C-23), 33.92 (CH₂, C-6), 33.58 (CH₂, C-7), 31.56 (CH₂, C-16), 30.71 (CH, C-8), 27.78 (3×CH₃, C-15′), 23.36 (CH₂, C-15), 20.56 (CH₃, C-19), 16.37 (CH₃, C-18).

### Mutual Prodrug 1 (5-{2-2[2(11,17-dihydroxy-10,13-dimethyl-10,13-dimethyl-3-oxo-6,7,8,9,10,11,12,13,14,15,16,17-dodecahydro-3H-cyclopenta[a]phenanthren-17-yl)-2-oxo-ethoxycarbonylmethyl]phenylazo}-2-hydroxy-benzoic acid)

To a solution of tert-butyl ester protected Mutual Prodrug 1 (0.1 g, 0.000155 mol) in dichloromethane (1 mL), trifluoroacetic acid (1 mL) was added reaction was left at room temperature during 4 hours. TLC analysis dichloromethane:ethyl acetate (50:50) shows completion and solvents and trifluoroacetic acid were blow off using nitrogen, to afford the product as an orange oil. The crude product was flash columned using dichloromethane:ethyl:acetate (50:50), to yield 0.078 g of the product as orange crystals (78%). mp: 154-156° C. MS: 665.2503 calculated mass 665.2475. IRᵥₘₐₓ (KBr): 3436.40 cm⁻¹, 1654.62 cm⁻¹, ¹H NMR 6 (CDCl₃): 8.48 (1H, s), 8.10 (1H, d, J=9.04 Hz), 7.75 (1H, d, J=7.52 Hz), 7.46 (4H, m), 7.08 (1H, d, J=9 Hz), 6.24 (1H, d, J=10 Hz), 6.00 (1H, s), 5.03 (2H, d, J=17.56 Hz), 4.35 (1H, s), 4.31 (2H, d, J=8.52 Hz), 2.68-0.8 (prednisolone envelope, 19H). ¹³C NMR 6 (MeOD): 205.18 (C, C-20, C═O), 187.15 (C, C-3, C═O), 172.99 (C, C-22, C═O), 171.82 (C, C-5), 164.11 (C, C-13′, C═O), 158.38 (C, C-10′), 156.35 (CH, C-1), 149.62 (C, C-2′), 144.93 (C, C-7′), 133.49 (C, C-1′), 133.36 (CH, C-5′), 130.90 (CH, C-6′), 129.97 (CH, C-8′), 127.39 (CH, C-2), 127.07 (CH, C-4′), 126.20 (CH, C-12′), 125.88 (CH, C-4), 120.57 (CH, C-11′), 117.01 (CH, C-3′), 114.64 (C, C-9′), 88.75 (C, C-17), 68.91 (CH, C-11), 67.95 (CH₂, C-21), 55.40 (CH, C-9), 50.91 (CH, C-14), MeOD residual peak is masking the peak of C, C-13, 44.19 (C, C-10), 38.19 (CH₂, C-12), 36.09 (CH₂, C-23), 33.70 (CH₂, C-6), 32.75 (CH₂, C-7), 31.31 (CH₂, C-16), 27.66 (CH, C-8), 22.95 (CH₂, C-15), 19.72 (CH₃, C-19), 15.36 (CH₃, C-18).

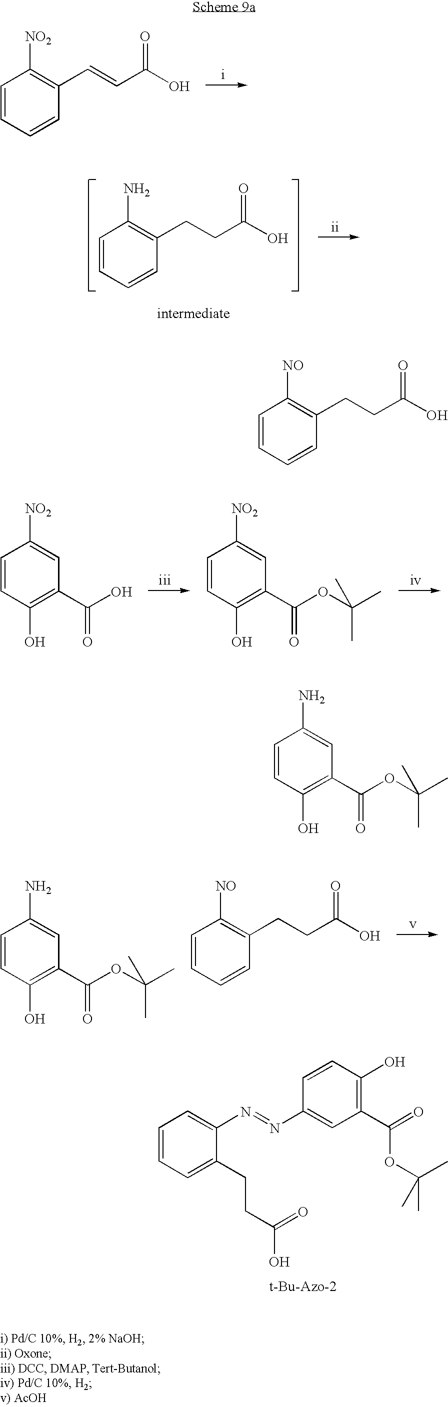

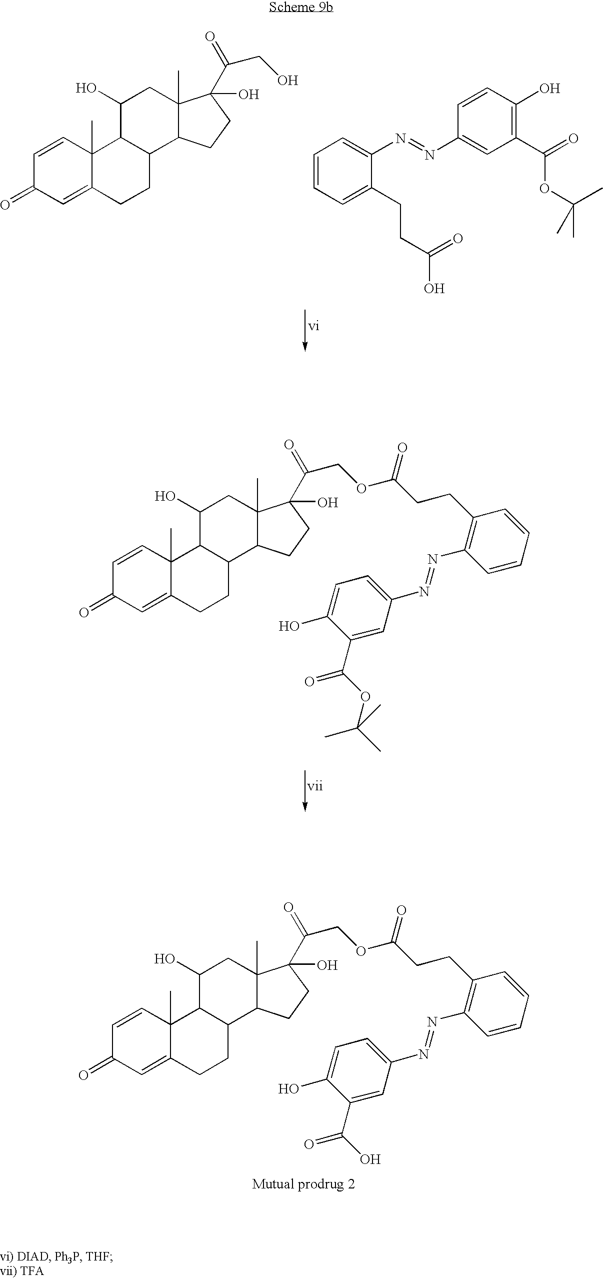

### 2-Nitrosophenyl Propionic Acid

To a solution of 2-nitrocinnamic acid (5 g, 0.0259 mol) in water 100 mL and 2 equivalents of 2% NaOH (103 mL), palladium 10% on charcoal was added on catalyst amount (5%, 0.25 g). The reaction mixture was stirred under hydrogen atmosphere at room temperature during 48 hours, after that time TLC analysis showed reaction completion (ethyl acetate). Reaction mixture was filtered over filter agent to remove the palladium 10% on charcoal and solvents were removed under reduced pressure. The crude 2-aminophenylpropionic was dissolved in distillated water 50 mL and dichloromethane 50 mL. Oxone® 2 equivalents (31.8 g, 0.0518 mol) were dissolved in distillated water 50 mL and added to the reaction mixture. At this point the reaction pH=4 so the 2-nitrosophenylpropionic acid is being transferred to the organic layer as soon as is formed. After 2 hours reaction mixture is transferred to a separate funnel and the organic layer collected, aqueous phase is washed 2×25 mL dichloromethane. The organic phase is collected and dry with Na₂SO₄. Solvents were removed under reduced pressure to afford 4.12 g of product as dark oil. The crude mixture was flash columned using dichloromethane:ethyl acetate (70:30) to yield 3.48 g product as brown crystals (67%): mp: 112-114° C., IRᵥₘₐₓ (KBr): 1699.29 (C═O) cm⁻¹. ¹H NMR δ (CDCl₃): 7.99 (1H, d, J=8 Hz), 7.65 (1H, t, J=7.52 Hz), 7.57 (1H, t, J=8.04 Hz), 7.43 (1H, m), 3.24 (2H, t, J=7.52 Hz), 2.81 (2H, t, J=7.52 Hz). ¹³C NMR δ (CDCl₃): 177.96 (C, C-9, C═O), 170.91 (C, C-2), 132.88 (CH, C-5), 131.70 (CH, C-6), 131.63 (C, C-1), 127.30 (CH, C-4), 124.56 (CH, C-3), 34.09 (CH₂, C-8), 27.61 (CH₂, C-7).

### 5-Nitrosalicylic tert-butyl ester

Dicyclohexylcarbodiimide (DCC) (6.2 g, 0.030 mol) dissolved in dry THF (50 mL), was added dropwise over 30 minutes to a stirred solution of 5-nitrosalicylic acid (5 g, 0.0273 mol) and 4-(dimethylamino)pyridine (DMAP) (3.3 g, 0.0273 mol) in dry tert-butanol (125 mL). The mixture was stirred overnight and filtered to remove dicyclohexylurea (DCU) formed; solvents were removed under reduced pressure to afford the product as yellow oil. This was flash columned using dichloromethane:ethyl acetate (90:10) to yield 5.29 g of product as pale yellow crystals (81%): mp: 58-60° C., IRᵥₘₐₓ (KBr): 3417.06 cm⁻¹, 2118.35 cm⁻¹, 1715.88 cm⁻¹, 1673.77 cm⁻¹. ¹H NMR δ (CDCl₃): 11.73 (1H, s), 8.63 (1H, d, J=2.52 Hz), 8.26 (1H, dd, J 9 and 2.48 Hz), 7.01 (1H, d, J=9.04 Hz), 1.61 (9H, s). ¹³C NMR δ (CDCl₃): 168.01 (C, C-2), 166.09 (C, C-7, C═O), 139.24 (C, C-5), 129.54 (CH, C-4), 126.19 (CH, C-6), 117.98 (CH, C-3), 112.95 (C, C-1), 84.45 (C, C-8), 27.57 (3×CH₃, C-9).

### 5-Aminosalicylic tert-butyl ester

To a solution of 5-nitrosalicylic tert-butyl ester (4.21 g, 0.0176 mol) in ethyl acetate (100 mL) palladium 10% on charcoal was added on catalyst amount (0.41 g, 10%). The reaction was stirred under hydrogen atmosphere during 3 hours. At that point TLC analysis (dichloromethane:ethyl acetate 60:40) showed completion. Reaction mixture was filtered over filter agent to remove the palladium 10% on charcoal and solvents were removed under reduced pressure, to yield 3.2 g of the product as pale green crystals (86%): mp: 48-50° C., IRᵥₘₐₓ (KBr): 3372.92 cm⁻¹, 1668.16 cm⁻¹, 1490.72 cm⁻¹. ¹H NMR δ (CDCl₃): 10.49 (1H, s), 7.12 (1H, d, J=2.52 Hz), 6.8 (2H, m), 3.5 (2H, NH₂), 1.61 (9H, s). ¹³C NMR δ (CDCl₃): 169.13 (C, C-7), 154.44 (C, C-2), 137.63 (C, C-5), 123.30 (CH, C-4), 117.57 (CH, C-3), 114.71 (CH, C-6), 113.24 (C, C-2), 82.12 (C, C-8), 27.74 (3×CH₃, C-9).

### 5-[2-(2-Carboxy-ethyl)phenylazo]-2-hydroxy-benzoic acid tert-butyl ester (t-Bu-Azo-2)

To a solution of 2-nitrosocinnamic acid (1.27 g, 0.0063 mol) in glacial acetic acid (20 mL), another solution of 5-aminosalicylic tert-butyl ester (1.32 g, 0.0063 mol) in glacial acetic acid (20 mL) is dropwise over 15 minutes. Reaction is left stirring 48 hours until completion by TLC dichloromethane:ethyl acetate (50:50). Solvents are removed under reduced pressure to afford the product as black oil. This was flash columned using dichloromethane as mobile phase to yield 1.68 g of the title compound as orange crystals (72%): mp: 86-88° C., IRᵥₘₐₓ (KBr): 1700.66 cm⁻¹, 1665.87 cm⁻¹. ¹H NMR δ (CDCl₃): 11.52 (1H, s), 8.46 (1H, s), 8.08 (1H, s), 7.72 (1H, s), 7.42 (3H, m), 7.13 (1H, s), 3.48 (2H, s), 2.82 (2H, s), 1.70 (9H, s). ¹³C NMR δ (CDCl₃): 179.00 (C, C-9, C═O), 169.18 (C, C-16, C═O), 163.79 (C, C-13), 149.57 (C, C-2), 145.09 (C, C-10), 139.21 (C, C-1), 130.54 (CH, C-5), 130.08 (CH, C-6), 127.65 (CH, C-11), 127.08 (CH, C-4), 126.97 (CH, C-15), 118.10 (CH, C-14), 115.07 (CH, C-3), 113.40 (C, C-12), 83.21 (C, C-17), 35.68 (CH₂, C-8), 27.74 (CH₃, C-18), 26.54 (CH₂, C-7).

### Tert-Butyl Protected Mutual Prodrug 2

To a solution of azo compound 2 (1.21 g, 0.0033 mol), prednisolone (1.1 eq, 1.18 g, 0.0033 mol) and triphenylphosphine (3 eq, 2.59 g, 0.0099 mol) in dry THF (50 mL), DIAD was added (3 eq, 1.9 mL, 0.0099 mol) dropwise over 12 minutes after the reaction temperature reach 40° C. Reaction was stirring at 40° C. during an hour and reaction is left overnight at room temperature under nitrogen atmosphere, TLC analysis (dichloromethane) shows completion. Solvents were removed under reduced pressure to afford the product as orange oil. This was flash columned using dichloromethane:ethyl acetate (50:50), after this column the triphenylphosphine oxide was removed and a second flash column is needed hexane (200 mL), hexane:ethyl acetate (70:30) to yield the product 1.60 g as orange crystals (40%): mp: 116-118° C., IRᵥₘₐₓ (KBr): 3435.94 cm⁻¹, 1724.53 cm⁻¹, 1658.49 cm⁻¹, 1615.49 cm⁻¹. ¹H NMR δ (CDCl₃): 8.40 (1H, d, J=2 Hz), 8.10 (1H, dd, J 8.52 and 2 Hz), 7.68 (1H, d, J=8.04 Hz), 7.44 (3H, m), 7.33 (1H, t, J=6.52 Hz), 7.09 (1H, d, J=8.52 Hz), 6.27 (1H, d, J=10.04 Hz), 6.01 (1H, s), 5.00 (1H, d, J=17.56 Hz), 4.88 (1H, d, J=17.56 Hz), 4.39 (1H, s), 3.49 (2H, t, J 9.52), 2.82 (2H, t, J=8.04 Hz), 2.66-0.9 (prednisolone envelope, 19H), 1.69 (9H, s). ¹³C NMR δ (CDCl₃): 204.83 (C, C-20, C═O), 186.46 (C, C-3, C═O), 172.51 (C, C-22, C═O), 170.73 (C, C-5), 169.08 (C, C-13′, C═O), 163.73 (C, C-10′), 156.77 (CH, C-1), 149.48 (C, C-2′), 145.09 (C, C-7′), 139.31 (C, C-1′), 130.55 (CH, C-5′), 130.00 (CH, C-6′), 129.26 (CH, C-8′), 127.04 (CH, C-2), 126.91 (CH, C-4′), 125.72 (CH, C-12′), 121.65 (CH, C-4), 118.18 (CH, C-11′), 114.99 (CH, C-3′), 113.29 (C, C-9′), 89.20 (C, C-17), 83.27 (C, C-14′), 69.56 (CH, C-11), 67.74 (CH₂, C-21), 54.90 (CH, C-9), 50.87 (CH, C-14), 47.22 (C, C-13), 43.84 (C, C-10), 38.96 (CH₂, C-12), 35.45 (CH₂, C-23), 33.83 (CH₂, C-6), 33.59 (CH₂, C-7), 31.61 (CH₂, C-16), 30.73 (CH, C-8), 27.75 (3×CH₃, C-15′), 26.66 (CH₂, C-24), 23.41 (CH₂, C-15), 20.54 (CH₃, C-19), 16.42 (CH₃, C-18).

### Mutual Prodrug 2 (5-(2-{2[2-(11,17-dihydroxy-10,13-dimethyl-10,13-dimethyl-3-oxo-6,7,8,9,10,11,12,13,14,15,16,17-dodecahydro-3H-cyclopenta[a]phenanthrene-17-yl)-2-oxo-ethoxycarbonyl]ethyl}-phenylazo}-2-hydroxy-benzoic acid)

To a solution of tert-butyl Azo 2 (0.9 g, 0.00126 mol) in dichloromethane (3 mL), trifluoroacetic acid 3 (mL) was added reaction was left at room temperature during 4 hours. TLC analysis dichloromethane:ethyl acetate (50:50) shows completion and solvents and trifluoroacetic acid were blow off using nitrogen, to afford the product as an orange oil. The crude product was flash columned using dichloromethane:ethyl acetate (50:50), to yield 0.7 g of the product as orange crystals (84%). mp: 176-178° C., IRᵥₘₐₓ (KBr): 3437.19 cm⁻¹, 1718.06 cm⁻¹, 1654.23 cm⁻¹, 1587.93 cm⁻¹. MS ¹H NMR δ (CDCl₃): 8.55 (1H, s), 8.00 (1H, dd, J 6.04 and 1.48 Hz), 7.65 (1H, d, J=5.28 Hz), 7.50 (1H, 6.76 Hz), 7.45 (1H, 4.52 Hz), 7.41 (1H, m), 7.34 (1H, t, J=4.76 Hz), 6.28 (1H, dd, J 6.76 and 1 Hz), 6.02 (1H, s), 4.90 (2H, d, J=29.84 Hz), 4.41 (1H, s), 3.50 (2H, t, J=5.04 Hz), 2.85 (2H, t, J=5 Hz), 2.67-0-93 (prednisolone envelope, 19H). ¹³C NMR 6 (MeOD): 205.37 (C, C-20, C═O), 187.17 (C, C-3, C═O), 173.05 (C, C-22, C═O), 172.05 (C, C-5), 164.03 (C, C-13′, C═O), 161.33 (C, C-10′), 158.41 (CH, C-1), 149.62 (C, C-2′), 144.93 (C, C-7′), 139.12 (C, C-1′), 129.85 (CH, C-6′), 128.63 (CH, C-8′), 126.47 (CH, C-2), 125.93 (CH, C-4′), 124.54 (CH, C-12′), 120.61 (CH, C-4), 117.65 (CH, C-11′), 116.80 (CH, C-3′), 114.51 (C, C-9′), 88.71 (C, C-17), 68.93 (CH, C-11), 67.48 (CH₂, C-21), 55.45 (CH, C-9), 50.94 (CH, C-14), MeOD residual peak is masking the peak of C, C-13, 44.23 (C, C-10), 38.37 (CH₂, C-12), 35.19 (CH₂, C-23), 33.72 (CH₂, C-6), 32.79 (CH₂, C-7), 31.33 (CH₂, C-16), 30.78 (CH, C-8), 26.26 (CH₂, C-24), 22.97 (CH₂, C-15), 19.73 (CH₃, C-19), 15.45 (CH₃, C-18).

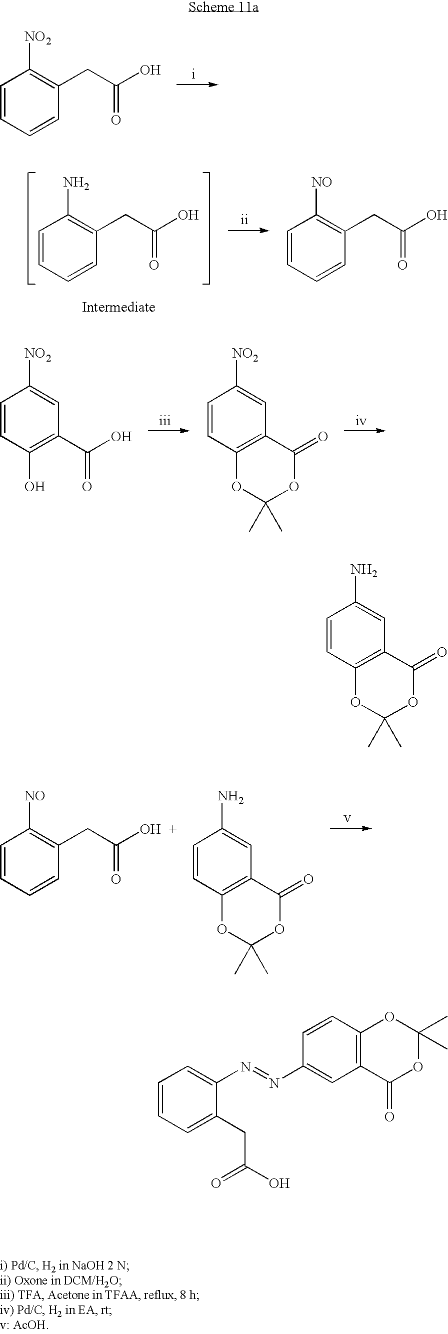

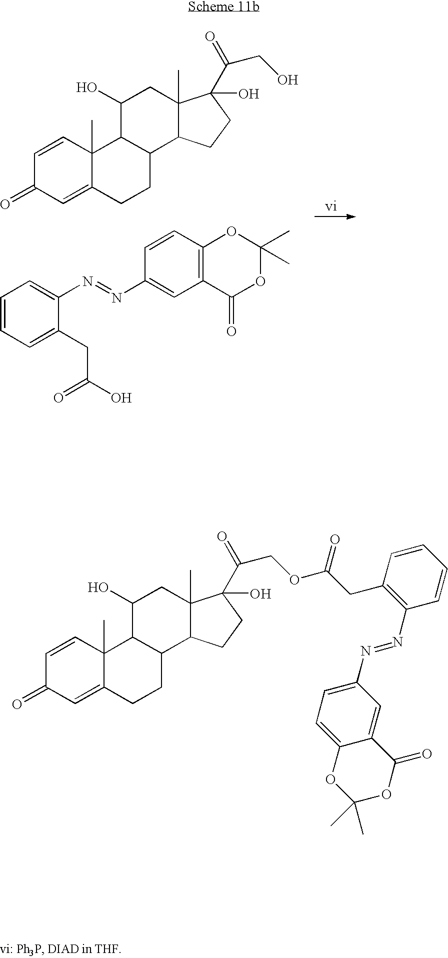

### 5-Nitrosalicylic acid dioxin-4-one

5-Nitrosalicylic acid (20 g, 109.2 mmol) was placed in a 500 ml round bottom flask equipped with a magnetic stirrer and a reflux condenser, 200 ml of trifluoroacetic acid was added followed by trifluoroacetic anhydride (45.54 ml, 327.7 mmol) and dry acetone (16.04 ml, 218.4 mmol). The reaction mixture was left at reflux for two hours. Further 8.02 ml of dry acetone was dropped to the boiling solution every hour (1 eq per hour) until reaction is complete within eight hours. The reaction is then concentrated under reduced pressure at about 55° C. bath temperature, toluene was added and remove three times to eliminate any trifluoroacetic acid traces and finally the solid residue was dried under vacuum for one hour at 45° C. The crude brownish solid was recrystallised twice from a mixture of acetone-petroleum ether (1:4), to yield 20.5 g of white off crystals (84.1%). (Alternatively flash chromatography can be used to purify the product when smaller scales are used using acetone-petroleum ether (1:4) or dichloromethane as a mobile phase): m.p. 92-94° C., IRᵥₘₐₓ (KBr): 1738.92 (C═O), 1616.23 and 745.94 (NO₂) cm⁻¹. ¹H NMR δ (CDCl₃): 8.88 (1H, d, J=2.51 Hz), 8.46 (1H, dd, J 6.52 and 3.01 Hz), 7.16 (1H, d, J=9.53 Hz), 1.80 (6H, s, 2×CH₃). ¹³C NMR δ (CDCl₃): 159.82 (C, C-7, C═O), 158.50 (C, C-4), 142.33 (C, C-1), 130.79 (CH, C-6), 125.57 (CH, C-2), 118.09 (CH, C-5), 112.93 (C, C-3), 107.36 (C, C-8), 25.45 (CH₃, C-9 and C-10).

### Acetonide-protected 5-aminosalicylic acid

To a solution of 5-nitrosalicylic acid dioxin-4-one (2 g, 8.96 mmol) in ethyl acetate (30 ml) at room temperature, was added palladium 10% on activated charcoal (0.5 g). The mixture was stirred under hydrogen atmosphere until reaction completed by TLC dichloromethane as mobile phase. The palladium was filtered through filter agent and solvents were removed under reduced pressure to yield 1.44 g of product as yellow crystals (83.2%): m.p. 122-124° C., IR_{Vmax} (KBr): 1710.37 (C═O), 3469.02 and 1324.22 (NH₂) cm⁻¹. MS: 216.0616 (M⁺⁺²³), 194.0814 (M⁺⁺¹). ¹H NMR δ (CDCl₃): 7.24 (1H, d, J=3.01 Hz), 6.91 (1H, dd, J 5.52 and 3.01 Hz), 6.79 (1H, d, J=9.04 Hz), 3.61 (2H, s, NH₂), 1.69 (6H, s, 2×CH₃). ¹³C NMR δ (CDCl₃): 161.16 (C, C-7, C═O), 148.15 (C, C-4), 141.36 (C, C-1), 123.38 (CH, C-6), 117.41 (CH, C-2), 113.54 (CH, C-5), 113.51 (C, C-3), 105.75 (C, C-8), 25.20 (CH₃, C-9 and C-10).

### 2-(2,2-Dimethyl-4-oxo-4H-benzo[1,3]dioxin-6-ylazo)-phenyl]-acetic acid (acetonide protected carrier)

To a solution of 2-nitrosophenyl acetic acid (0.5 g, 2.58 mmol) in glacial acetic acid (25 ml) another solution of 5-aminosalicylic acid dioxin-4-one (0.42 g, 2.58 mmol) in glacial acetic acid (25 ml) was added. The reaction mixture was stirred vigorously during forty eight hour under a nitrogen atmosphere; reaction completion was checked by TLC using dichloromethane-ethyl acetate (50:50) as mobile phase. The solvent was removed under reduced pressure and toluene added two times to eliminate any of acetic acid traces to afford an orange crude product. This was purify by flash chromatography using dichlormethane-ethyl:acetate (70:30) as mobile phase to yield 0.78 g of product as orange crystals (89%): m.p. 124-126° C., IR_{Vmax} (KBr): 1734.84 and 1698.18 (C═O) cm⁻¹. MS: 363.0950 (M++²³). ¹H NMR δ (CDCl₃): 8.52 (1H, d, J=2.51 Hz), 8.05 (1H, dd, J 8.54 and 2.51 Hz), 7.77 (1H, d, J=7.53 Hz), 7.47 (1H, t, J=6.57 Hz), 7.43 (2H, m), 7.05 (1H, d, J=9.03 Hz), 4.16 (s, 2H), 1.80 (6H, s, 2×CH₃). ¹³C NMR δ (CDCl₃): 177.67 (C, C-8, C═O), 160.42 (C, C-15, C═O), 157.85 (C, C-12), 149.66 (C, C-1), 147.86 (C, C-9), 133.78 (C, C-2), 131.55 (CH, C-4), 131.45 (CH, C-3), 128.93 (CH, C-5), 128.39 (CH, C-14), 126.64 (CH, C-10), 118.14 (CH, C-6), 116.07 (CH, C-13), 113.61 (C, C-11), 106.90 (C, C-16), 37.28 (CH₂, C-7), 25.86 (CH₃, C-17 and C-18).

### Acetonide Protected Mutual Prodrug 1

To a solution of 2-(2,2-dimethyl-4-oxo-4H-benzo[1,3]dioxin-6-ylazo)-phenyl]-acetic acid (1 g, 5.16 mmol) in dry tetrahydrofuran (30 ml), prednisolone (2.23 g, 6.19 mmol) was added followed by triphenylphosphine (4.11 g, 15.5 mmol) at room temperature. The reaction mixture was stirred under nitrogen atmosphere and the temperature was raised until 40° C. At this point diisopropylazodicarboxylate 95% (DIAD) (3.2 ml, 15.5 mmol) was dropwise over twelve minutes. The mixture was stirred over two hours and volatiles were removed under reduced pressure to afford the product as crude black oil. Two flash chromatography columns (dichloromethane-ethyl acetate 1:1 and Hexane-ethyl acetate 80:20) were needed to yield 2.21 g of the pure product as orange crystals (63%). m.p. 136-138° C., IR_{Vmax} (KBr): 1655.56 (C═O), 1724.45 (C═O), 3462.94 (O—H) cm⁻¹. MS 705.2784 (M++²³). ¹H NMR δ (CDCl₃): 8.43 (1H, d, J=2.01 Hz), 8.18 (1H, dd, J 9.03 and 2.51 Hz), 7.73 (1H, d, J=8.03 Hz), 7.43 (2H, m), 7.38 (1H, m), 7.25 (1H, d, J=10.04 Hz), 7.11 (1H, d, J=8.53 Hz), 6.21 (1H, d, J=10.04 Hz), 5.95 (1H, s), 5.01 (1H, d, J=17.56 Hz), 4.92 (1H, d, J=17.57 Hz), 4.31 (1H, s), 4.22 (2H, s), 2.64-0.84 Prednisolone envelope, 19H), 1.75 (6H, s, 2×CH₃). ¹³C NMR δ (CDCl₃): 204.67 (C, C-20, C═O), 186.36 (C, C-3, C═O), 171.35 (C, C-22, C═O), 170.53 (C, C-13, C═O), 160.46 (C, C-5), 157.30 (C, C-10′), 156.67 (CH, C-1), 149.24 (C, C-2′), 147.64 (C, C-7′), 134.04 (C, C-1′), 131.66 (CH, C-6′), 131.28 (CH, C-12′), 131.22 (CH, C-4′), 127.88 (CH, C-2), 127.00 (CH, C-5′), 123.35 (CH, C-8′), 121.65 (CH, C-4), 117.67 (CH, C-11′), 115.24 (CH, C-3′), 113.17 (C, C-9′), 106.59 (C, C-14′), 89.18 (C, C-17), 69.44 (CH, C-11), 68.19 (CH₂, C-21), 54.88 (CH, C-9), 50.84 (CH, C-14), 47.15 (C, C-13), 43.80 (C, C-10), 38.65 (CH₂, C-12), 37.02 (CH₂, C-23), 33.70 (CH₂, C-6), 33.61 (CH₂, C-7), 31.57 (CH₂, C-16), 30.76 (CH, C-8), 25.41 (CH₃, C-15′ and C-16′), 23.39 (CH₂, C-15), 16.24 (CH₃, C-19), 14.81 (CH₃, C-18).

### 2-(2,2-Dimethyl-4-oxo-4H-benzo[1,2]dioxin-6-ylazo)-phenyl]-propionic acid

To a solution of 2-nitrosophenylpropionic acid (1 g, 4.97 mmol) in glacial acetic acid (40 ml) another solution of 5-aminosalicylic acid dioxin-4-one (0.9 g, 4.97 mmol) in glacial acetic acid (40 ml) was added. The reaction mixture was vigorously stirred for forty eight hours under nitrogen atmosphere; reaction completion is checked by TLC using dichloromethane-ethyl acetate (50:50) as a mobile phase. The solvent was removed under reduced pressure and toluene added two times to eliminate any of acetic acid traces to afford an orange crude product. This was purified by flash chromatography using dichlormethane-ethyl:acetate (70:30) as mobile phase to yield 1.17 g of product as orange crystals (68%): m.p. 108-110° C., IR_{Vmax} (KBr): 1703.48 and 1740.04 (C═O) cm⁻¹. MS 377.1102 (M⁺⁺²³), ¹H NMR δ (CDCl₃): 8.57 (1H, d, J=2 Hz), 8.17 (1H, dd, J 6.52 and 2.51 Hz), 7.72 (1H, d, J=7.53 Hz), 7.43 (2H, m), 7.15 (1H, d, J=9.03 Hz), 3.50 (2H, t, J 8.03 and 7.53 Hz), 2.79 (2H, t, J 8.03 and 7.53 Hz), 1.81 (6H, s, 2×CH₃). ¹³C NMR δ (CDCl₃): 176.97 (C, C-9, C═O), 160.11 (C, C-16, C═O), 157.37 (C, C-13), 149.39 (C, C-1), 147.64 (C, C-10), 139.80 (C, C-2), 131.16 (CH, C-4), 130.15 (CH, C-3), 129.01 (CH, C-5), 126.99 (CH, C-15), 125.60 (CH, C-11), 117.75 (CH, C-6), 115.20 (CH, C-14), 113.32 (C, C-12), 106.51 (C, C-17), 35.46 (CH₂, C-8), 26.43 (CH₂, C-7), 25.48 (CH₃, C-17 and C-18).

### Acetonide Protected Mutual Prodrug 2

To a solution of a2-(2,2-dimethyl-4-oxo-4H-benzo[1,2]dioxin-6-ylazo)-phenyl]-propionic acid (0.2 g, 0.56 mmol) in dry tetrahydrofuran (10 ml), prednisolone (0.2 g, 0.85 mmol) was added followed by triphenylphosphine (0.44 g, 1.68 mmol) at room temperature. The reaction mixture was stirred under nitrogen atmosphere and the temperature was raised until 40° C. At this point diisopropylazodicarboxylate 95% (DIAD) (0.35 ml, 1.68 mmol) was dropwise over twelve minutes. The mixture was stirred over two hours and volatiles were removed under reduced pressure to afford the product as crude black oil. Two flash chromatography columns (dichloromethane-ethyl acetate 1:1 and Hexane-ethyl acetate 80:20) were needed to yield 0.26 g of the pure product as orange crystals (68%). m.p. 130-132° C., IR_{Vmax} (KBr): 3437.13, 1743.91, 1656.48, 1615.31 cm⁻¹. ¹H NMR δ (CDCl₃): 8.47 (1H, d, J=2.01 Hz), 8.16 (1H, dd, J 8.53 and 2.01 Hz), 7.67 (1H, d, J=8.03 Hz), 7.38 (2H, m), 7.28 (2H, m), 7.10 (1H, d, J=8.54 Hz), 6.23 (1H, d, J=10.03 Hz), 5.97 (1H, s), 5.08 (1H, d, J=17.57 Hz), 4.94 (1H, d, J=17.56 Hz), 4.42 (1H, s), 3.45 (2H, t, J 6.52), 2.79 (2H, t, J=8.03 Hz), 2.68-0.91 (Prednisolone envelope, 19H), 1.75 (6H, s, 2×CH₃). ¹³C NMR δ (CDCl₃): 204.92 (C, C-20, C═O), 186.44 (C, C-3, C═O), 172.37 (C, C-22, C═O), 170.68 (C, C-13, C═O), 160.45 (C, C-5), 157.25 (C, C-10′), 156.79 (CH, C-1), 149.14 (C, C-2′), 147.61 (C, C-7′), 139.99 (C, C-1′), 131.23 (CH, C-6′), 131.00 (CH, C-12′), 130.05 (CH, C-4′), 127.02 (CH, C-2), 126.93 (CH, C-5′), 123.74 (CH, C-8′), 121.64 (CH, C-4), 117.77 (CH, C-11′), 115.16 (CH, C-3′), 113.18 (C, C-9′), 106.57 (C, C-14′), 89.18 (C, C-17), 69.54 (CH, C-11), 67.77 (CH₂, C-21), 54.92 (CH, C-9), 50.88 (CH, C-14), 47.14 (C, C-13), 43.86 (C, C-10), 38.85 (CH₂, C-12), 35.89 (CH₂, C-23), 33.75 (CH₂, C-6), 33.61 (CH₂, C-7), 31.62 (CH₂, C-16), 30.78 (CH, C-8), 26.93 (CH₃, C-15′ and C-16′), 25.42 (CH₂, C-24), 23.45 (CH₂, C-15), 16.35 (CH₃, C-19), 14.81 (CH₃, C-18).

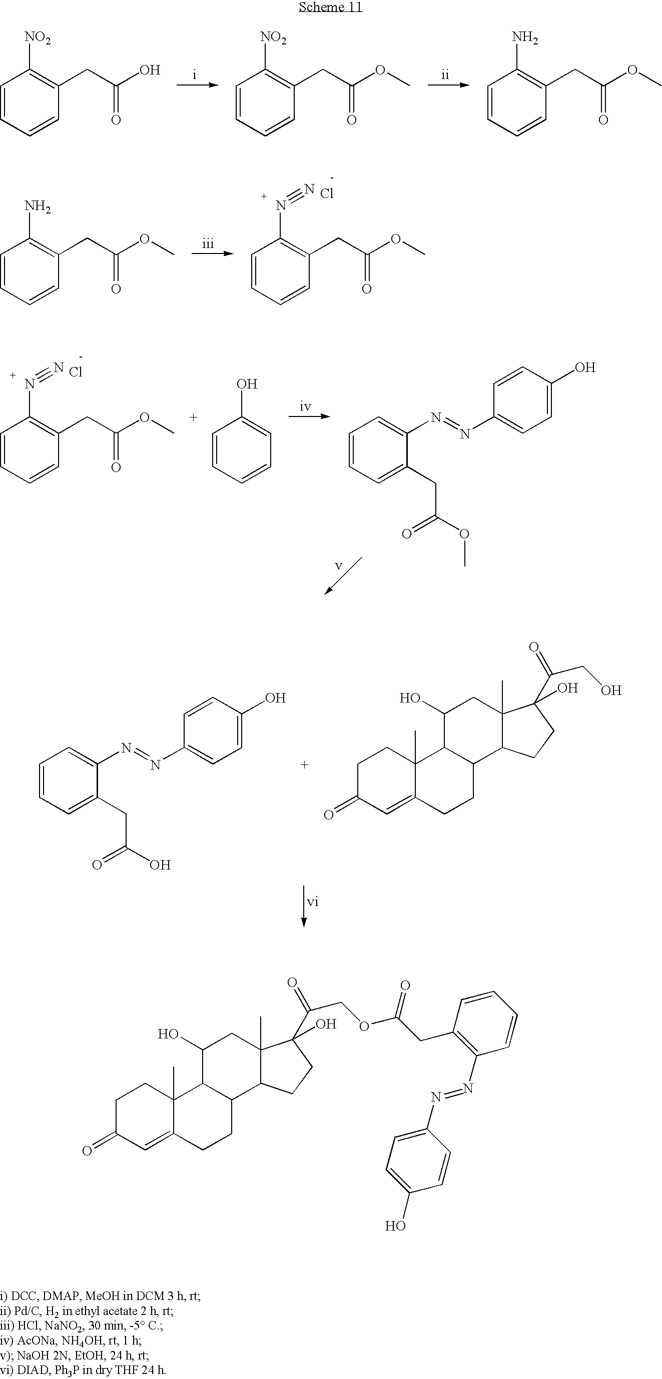

### 2-Nitrophenyl acetic acid methyl ester

To a solution of 2-nitrophenyl acetic acid (5 g, 0.0276 mol) in DCM (50 mL) was added DMAP (1 eq, 3.37 g) followed by DCC (1 eq, 5.63 g) and methanol (5 eq, 5.58 mL) was added last. The reaction mixture was stirred 2-3 h, the TLC shows when the reaction was completed. After that the reaction was filtered to remove the DCU formed and the filtrate was washed with 0.1N HCl (2×25 mL), saturated aqueous sodium bicarbonate solution (2×25 mL) and water (2×25 mL), dried over sodium sulfate and the solvent remove under reduced pressure. The crude ester was purified by flash chromatography (DCM:EA 60:40), to yield the product as a yellow oil 4.8 g (90%): ¹H-NMR (400 MHz, CDCl₃), δ: 7.94 (1H, d, J=8 Hz), 7.4 (1H, t, J=7.2 Hz), 7.3 (1H, t, J=7.6 Hz), 7.25 (1H, d, J=7.6 Hz), 3.89 (2H, s), 3.59 (3H, s). ¹³C-NMR (400 MHz, CDCl₃) δ: 170.3 (C═O, C-8), 148.2 (C-1), 133.0 (C-4), 132.8 (C-3), 129.2 (C-2), 128.0 (C-5), 124.5 (C-6), 51.4 (C-9), 38.8 (C-7).

### 2-aminophenyl acetic acid methyl ester

The 2-nitrophenyl acid methyl ester (1 g, 5.13 mmol) was dissolved in 20 mL of EA and 0.3 g of Pd/C 10% was suspended into the solution, the reaction mixture was under hydrogen atmosphere at room temperature. After 2 h the reaction was completed by TLC analysis (DCM:EA 60; 40) the Pd/C 10% was removed by filtration and the solvent by reduced pressure; to yield a colorless oil 0.67 g (67%).

### 2-(4-Hydroxy-phenylazo)-phenyl-acetic acid methyl ester

The 2-aminophenyl acid methyl ester (3 g, 0.018 mol) was dissolved in water (40 mL) with concentrated HCl (9 mL) and the solution was cooled in an ice/acetone bath to −5° C., afterward sodium nitrite (1.32 g, 0.019 mol) in 1 mL of water was added dropwise. At that point the diazonium salt was formed and the solution took a pale yellow color. A spatula of urea was added to neutralize the excess of sodium nitrite (oxidant) and the diazonium salt solution was added dropwise into a solution of phenol (1.68 g, 0.018 mol), sodium acetate (2.88 g, 0.036 mol) and ammonia solution (5 mL) in water (40 mL), which was at −5° C., after 5 minutes. After the addition was done glacial acetic acid (eq, 5 mL) was added and the reaction mixture was filtered. The filter cake was dissolved in EA and dried over sodium sulfate, the solvent removed under reduced pressure and the product purified by flash chromatography (DCM 100 mL, DCM:EA 80:20) to yield 2.7 g (56%): m.p. 158-160° C., IRᵥₘₐₓ (KBr): 3221.84 (OH), 1695.48 (C═O) cm⁻¹. ¹H-NMR (400 MHz, Acetone) δ: 9.14 (1H, s), 7.86 (2H, d, J=8.50 Hz), 7.72 (1H, d, J=8 Hz), 7.45 (3H, m, J=7.52 Hz), 7.03 (2H, d, J=8.50 Hz), 4.16 (2H, s), 3.62 (3H, s). ¹³C-NMR (400 MHz, Acetone) δ: 204.91 (C═O, Acetone), 171.22 (C═O, C-8), 160.29 (C, C-4′), 149.64 (c, C-2), 146.20 (C, C-1′), 134.13 (CH, C-6), 131.13 (CH, C-5), 129.97 (C, C-1), 127.42 (CH, C-4), 124.65 (CH, C-3, C-6′, C-2′), 115.38 (CH, C-5′), 114.73 (CH, C-3′), 50.65 (CH₃, C-9), 36.41 (CH₂, C-7), 28.52 (Acetone, CH₃).

### 2-(4-Hydroxy-phenylazo)-phenyl-acetic acid

The ester (0.266 g, 1 mmol) was dissolved in 10 mL of ethanol under nitrogen atmosphere and 2N NaOH (2 mL) was added into the mixture and stirred over night with shielding from light. The reaction mixture was poured in 1N HCl solution and extracted with EA. The organic phase was washed with water and brine and dried over magnesium sulfate. The solvent removed under reduced pressure and the product recrystallised with EA: n-hexane to yield the product as orange crystals. 0.2 g (70%): m.p. 138-140° C., IRᵥₘₐₓ (KBr): 3280.04 (OH), 1721.07 (C═O), 1655.90 (C═O) cm⁻¹. ¹H-NMR (400 MHz, Acetone) δ: 10.71 (1H, s), 9.13 (1H, s), 7.88 (2H, d, J=8.52 Hz), 7.71 (1H, d, J=7.52 Hz), 7.45 (3H, m, J=8.04 Hz), 7.01 (2H, d, J=8.52 Hz), 4.16 (2H, s). ¹³C-NMR (400 MHz, Acetone) δ: 204.92 (C═O, Acetone), 171.65 (C═O, C-8), 160.20 (C, C-4′), 149.73 (C, C-2), 134.49 (C, C-1′), 131.18 (CH, C-5), 129.91 (CH, C-6, C-1), 127.28 (CH, C-4), 124.69 (CH, C-3, C-6′, C-2′), 115.32, (CH, C-5′) 114.63 (CH, C-3′), 36.23 (CH₂, C-7), 28.51 (Acetone, CH₃).

### [2-(4-Hydroxy-phenylazo)-phenyl]acetic acid 2-(11,17-dihydroxy-10,13-dimethyl-3 oxo-2,3,6,7,8,9,10,11,12,13,14,15,16,17-tetradecahydro-1H-cyclopenta[α]phenanthren-17-yl)-2-oxo-ethyl ester Phenol Azoprodrug

The azo carrier (0.5 g, 0.0019 mol) was added to a solution of hydrocortisone (0.34 g, 0.0009 mol) and triphenylphosphine (0.74 g, 0.0028 mol) in dry THF, under nitrogen atmosphere the temperature was raised to 40° C. and diisopropylazodicarboxylate 95% (DIAD) (0.57 g, 0.0028 mol) was added dropwise over 12 minutes with vigorous stirring. The mixture was stirred at 40° C. over 1 h and reaction was left overnight at room temperature under nitrogen atmosphere. The volatiles were removed under reduced pressure to afford the product as orange oil. This was flash columned using dichloromethane:ethyl acetate (50:50), after that column the triphenylphosphine oxide was removed and a second flash column was needed hexane (200 mL), hexane:ethyl acetate (70:30) to yield the azoprodrug 0.23 g as orange crystals (42%): m.p. 122-124° C., IRᵥₘₐₓ (KBr): 3437.13 (OH), 1721.07 (C═O), 1655.90 (C═O) cm⁻¹. ¹H-NMR (400 MHz, Acetone) δ: 7.76 (2H, d, J=8.53 Hz), 7.65 (1H, d, J 7.03), 7.33 (3H, m), 6.85 (2H, d, J=8.53 Hz), 5.59 (1H, s), 4.90 (1H, d, J=17.57 Hz), 4.74 (1H, d, J=17.06 Hz), 4.18 (2H, s), 2.60-0.77 (Hydrocortisone envelope, 24H).

### Intramolecular Lactamization Kinetic Studies

Release of steroid in vivo from the prodrugs hinges upon the cyclization of the amino steroid product of azo-reductase activity. The cyclization rates of the amino derivatives of cortisone A and B were monitored in aqueous solution at various pH values and at 37° C. by measuring remaining compound by HPLC along with the evolution of cortisone.

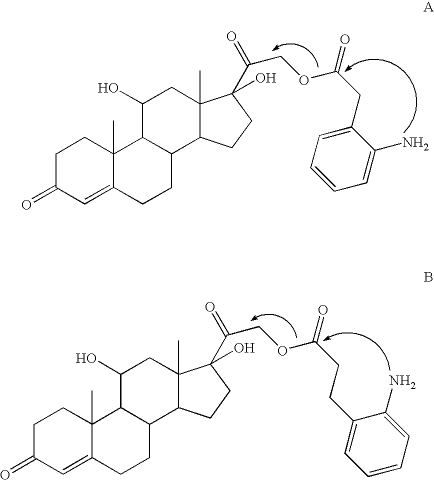

The first order rate constant for disappearance can be obtained from a plot of the natural log of remaining ester versus time. The half-life can be calculated (T_{1/2}=0.693/k_{obs}). The calculated half-life of prodrug A is 2.2 hours; the calculated half-life of prodrug B is 1.4 hours.

### Biological Studies

### Materials and Methods

Prednisolone and DSS were purchased from Sigma-Aldrich laboratories.

BALB/c strain mice were from the Bioresources Unit (Trinity College Dublin). Mice were housed in individually ventilated and filtered cages under positive pressure (Tecniplast, Nothants, UK). Food and water were supplied ad libitum. All animal experiments were performed in compliance with Irish Department of Health and Children regulations and approved by the Trinity College Bioresources ethical review board.

### Statistical Analysis

All in vivo experiments were performed with six mice per group. Difference between groups was analyzed by Student's t-test. Colitis scores were analyzed by Mann-Whitney test. P values <0.05 were considered significant. Results on the DIA, C/B and T/B MPO were expressed as means±S.E.

### Preparation of Inflammatory Bowel Disease Model Mice

DSS (35-50,000 kDa; MP Biomedicals, OH) was dissolved in the drinking water of the mice. Fresh DSS solution was provided every second day. BALB/c mice were exposed to 5% DSS for six days. The mice were checked every day for morbidity and weight recorded. Induction of colitis was determined by weight loss, fecal blood and, upon autopsy, length of colon. Blood in feces was detected using a Hemdetect occult blood detection kit (Dipro, Austria).

To quantify induction of colitis, a disease activity index (DAI) was determined based on previous studies of DSS-induced colitis (Cooper 1993). DAI was calculated for each mouse daily based on body weight loss, occult blood and stool consistency/diarrhea. A score of 1-4 was given for each parameter, with a maximum DAI score of 12. Score 0, No weight loss, normal stool, no blood; Score 1, 1-3% weight loss; Score 2, 3-6% weight loss, loose stool, blood visible in stool; Score 3, 6-9% weight loss; Score 4, <9% weight loss, diarrhea, gross breeding. Loose stool was defined as the formation of a stool that readily becomes paste upon handling. Diarrhea was defined as no stool formation. Gross bleeding was defined as fresh blood on fur around the anus with extensive blood in the stool,

### Colon Histology

At autopsy the length of the colon was measured and a 1 cm section of colon was fixed in 10% formaldehyde-saline. H&E-stained sections were grading based on a scoring system modified from a previous study (Siegmund 2001). Histology scoring was performed in a blinded fashion. A combined score of inflammatory cell infiltration and tissue damage was determined as follows: Cell infiltration: Score 0, Occasional inflammatory cells in the LP; 1, Increased infiltrate in the LP predominantly at the base of crypts; 2, Confluence of inflammatory infiltrate extending into the mucosa; 3, Trans-mural extension of infiltrate. Tissue Damage Score 0, No mucosal damage; 1, Partial (up to 50%) loss of crypts in large areas; 2, Partial to total 50-100% loss of crypts in large areas, epithelium intact; 3, Total loss of crypts in large areas and epithelium lost.

### Drug Efficacy

The DSS induced colitis mice weighting about 18-20 grams were allowed 5% DSS water ad libitum and 100 μL of prednisolone and prodrug 2 in 1% solution cremophor and ethanol in water suspension (equivalent to 5 mg of prednisolone per Kg or mouse body weight) was orally administrated, twice a day (every twelve hours) to the colitis mice. Six days after the prednisolone and prodrugs treatments, the mice were sacrificed by cervical dislocation. The distal colon segment and the thymus were removed and washed to remove contents of the colon.

One centimetre of the distal colon segment and the fresh weight of the thymus were measured to obtain the ratios of distal colon weight and thymus weight to body weight (C/B and T/B respectively). The damage of the colon specimens were scored as describe in materials and methods. The distal segments were frozen by liquid nitrogen kept at −80° C. until assayed for myeloperoxidase activity (MPO).

### Results

Anti-inflammatory effects were studied by comparison of the distal colon length between healthy mice, untreated mice, prednisolone treated mice and prodrug treated mice. IBD causes shortering of the colon. Thus anti-inflammatory effects the less damage was observed on the colon tissue and less reduction was cause on the distal colon length.

Systemic side effects were study by comparison of the thymus weight and body weight ratio (Hideki Yano 2002) between health mice, untreated mice, prednisolone treated mice and prodrug treated mice, due to the fact that an increase on the concentration of steroid in the bloodstream provokes suppression on the activity of the thymus and the consequent reduction of its weight.

As shown in FIG. 13, a profile of the DAI score from colon segment of DSS-induced colitis mice treated at 5 mg/Kg dosage, the subjective assessment of bleeding and weight loss score increases towards the threshold value in the vehicle control group but not in the treatment groups.

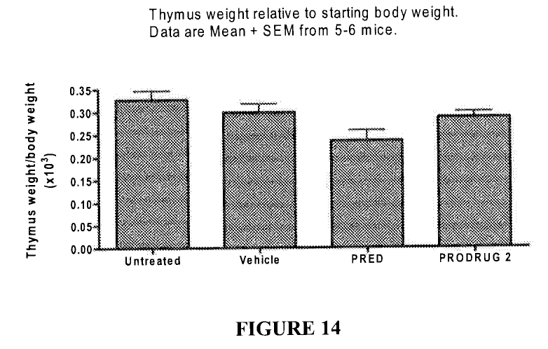

As shown in FIG. 14, a profile of colon length of DSS-induced colitis mice treated at 5 mg/Kg dosage, Prodrug 2 Group had significantly longer colon than the PRED (P<0.0243) group. (P values are Student's t-test of difference in colon length versus vehicle group.)

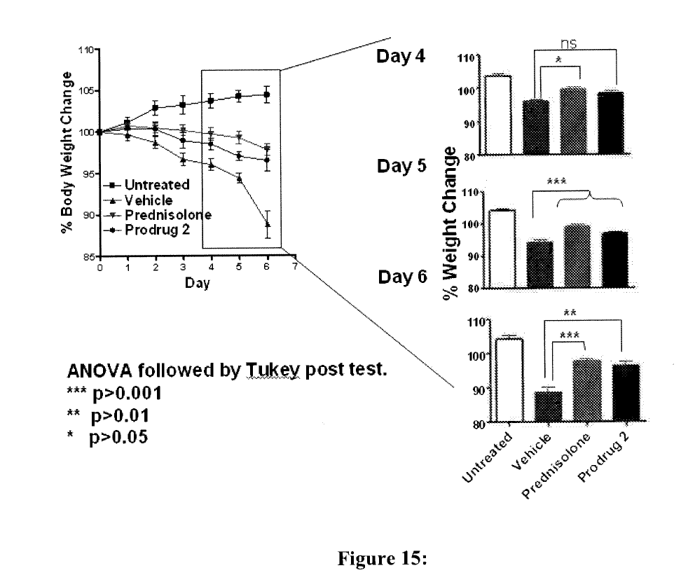

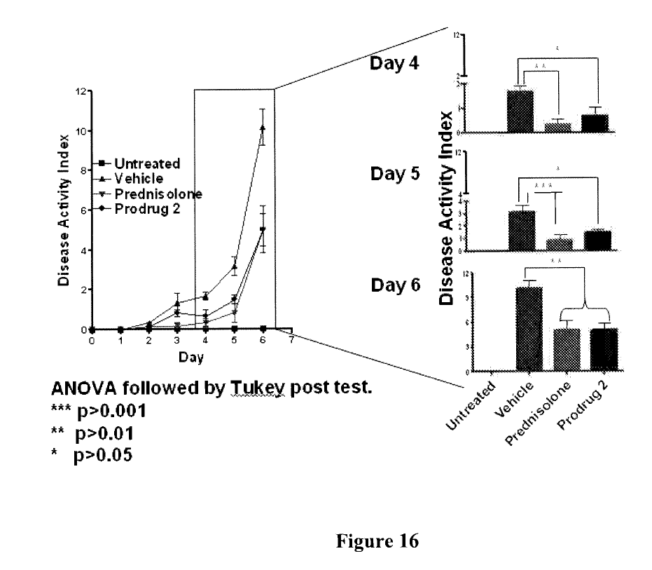

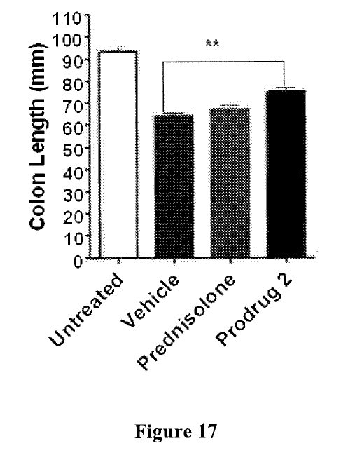

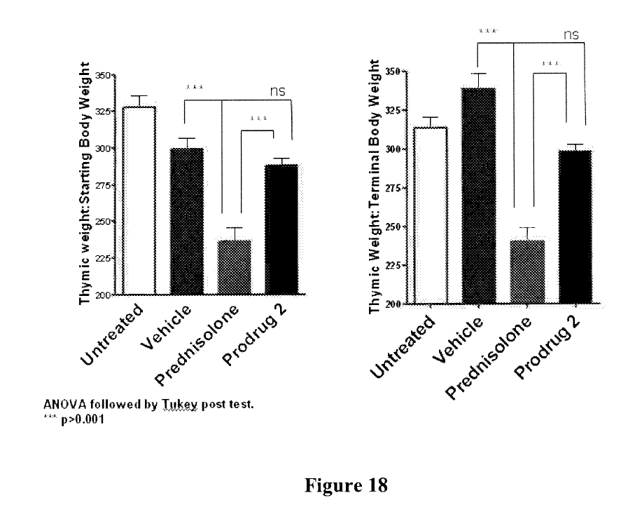

As shown in FIG. 15, a profile of thymus weight body weight ratios (T:BW) of healthy mice (Untreated) and DSS-induced colitis mice treated at 5 mg/Kg dosage, the PRED group had significant reduction in thymus:BW ratio versus the Untreated mice (P<0.05). The mean thymus weight in the prodrug treatment groups was not statistically different to the untreated mice.

### REFERENCES

a) Friend D. R. and Chang G. W., 1985. Drug glycosides: potential prodrugs for colon specific drug delivery. J. Med. Chem. 28, 51-57; b) Fedorak R. N., Cui N., Friend D. R., Madsen K. L., Empey L. R., 1995. A novel colon-specific steroid prodrug enhances sodium chloride absorption in rat colitis. Am. J. Physiol. 269 (2 Pt 1):G210-8. PMID: 7653560; c) Fedorak R. N., Haeberlin B., Empey L. R., Cui N., Nolen H. 3rd, Jewell L. D., Friend D R., 1995. Colonic delivery of dexamethasone from a prodrug accelerates healing of colitis in rats without adrenal suppression. Gastroenterology. 108(6):1688-99. PMID: 7768373; d) Friend D. R., Chow J. J., Chang G. W., 1990. Effect of antibiotic pretreatment on glycoside/glycosidase-based colonic drug delivery. Drug Des Deliv. 6(4):311-8. PMID: 2083030; e) McLeod A D, Fedorak R N, Friend D R, Tozer T N, Cui N., 1994. A glucocorticoid prodrug facilitates normal mucosal function in rat colitis without adrenal suppression. Gastroenterology. 106(2):405-13. PMID: 7507873; f) Nolen H. 3rd, Fedorak R. N., Friend D. R., 1995. Budesonide-beta-D-glucuronide: a potential prodrug for treatment of ulcerative colitis. J Pharm Sci. 84(6):677-81. PMID: 7562403. g) Tozer T. N., Rigod J., McLeod A. D., Gungon R., Hoag M. K., Friend D. R., 1991. Colon-specific delivery of dexamethasone from a glucoside prodrug in the guinea pig. Pharm Res. 8(4):445-54.PMID: 1871038.
Bai J. P., Chang L. L., Guo J. H., 1995. Targeting of peptide and protein drugs to specific sites in the oral route. Crit Rev Ther Drug Carrier Syst; 12(4):339-71.
Haeberlin B., Rubas W., Nolen H. W. 3rd, Friend D. R., 1993. In vitro evaluation of dexamethasone-beta-D-glucuronide for colon-specific drug delivery. Pharm Res. 11, 1553-62. PMID: 8290467.
Hu Z., Kimura G., Mawatari S., Shimokawa T., Yoshikawa Y., Takada K., 1998. New preparation method of intestinal pressure-controlled colon delivery capsules coating machine and evaluation in beagle dogs. J Control. Release 56, 293-302, 1922-4.
Komiya, I., Park, J. Y., Kamani, A., Ho, N. F. H. 1980, Int. J. Pharm. 4:249.
Leopold C. S., Friend D. R., 1995. In vivo pharmacokinetic study for the assessment of poly(L-aspartic acid) as a drug carrier for colon-specific drug delivery. J Pharmacokinet Biopharm. 23(4):397-406. PMID: 8882747.
McLeod A. D., Tolentino L., Tozer T. N., 1994. Glucocorticoid-dextran conjugates as potential prodrugs for colon-specific delivery: steady-state pharmacokinetics in the rat. Biopharm Drug Dispos. 15(2): 151-61. PMID: 7516722.
Sinha V. R., Kumria R., 2001. Colonic drug delivery: prodrug approach. Pharm. Res. 18, 557-564. Stewart B. H., Amidon G. L., Brabec R. K., 1986. Uptake of prodrugs by rat intestinal mucosal cells: mechanism and pharmaceutical implications. J Pharm Sci. 75(10):940-5. PMID: 3795023.
Yang L., Chu J. S., Fix J. A., 2002. Colon-specific drug delivery: new approaches and in vitro/in vivo evaluation. Int. J. Pharm. 235, 1-15.
Yano H, Hirayama F, Kamada M, Arima H, Uekama K., 2002. Colon-specific delivery of prednisolone-appended alpha-cyclodextrin conjugate: alleviation of systemic side effect after oral administration. Control Release. 79(1-3):103-12. PMID: 11853922.
Cooper, H. S., S. N. Mrurthy, R. S. Shah, and D. J. Sedegran. (1993). “Clinicopathologic study of dextan sulfate sodium experimental murine colitis.” Lab Invest 69: 238-249.
Elson, C. O., R. B. Sartor, G. S. Tennyson, and R. H. Riddell. (1995). “Experimental models of inflammatory bowel disease.” Gastroenterology 109: 1344-1367.
Hideki Yano, F. H., Makoto Kamada, Hidetoshi Arima, Kaneto Uekama (2002). “Colon-specific delivery of prednisolone-appended alfa-cyclodextrin conjugate: alleviation of systemic side effect after oral administration.” Journal of Controlled Release 79: 103-112.
Okayasu, I., S. Hatakeyama, M. Yamada, T. Ohkusa, Y. Inagaki, and R. Nakaya. (1990). “A novel method in the induction of reliable experimental acute and chronic ulcerative colitis in mice.” Gastroenterology 98: 694-702.
Siegmund, B., G. Fantuzzi, F. Rieder, F. Gamboni-Robertson, H. A. Lehr, G. Hartmann, C. A. Dinarello, S. Endres, and A. Eigler. (2001). “Neutralization of interleukin-18 reduces severity in murine colitis and intestinal IFN-gamma and TFN-alpha production.” Am. J. Physiol. Regul. Integr. Comp. Physiol 281: 1264-1273.
Verdu, E. F., P. Bercik, B. Cukrowska, M. A. Farre-Castany, H. Bouzourene, E. Saraga, A. L. Blum, I. Corthesy-Theulaz, H. Tlaskalora-Hogenova, and P. Michetti (2000). “Oral administration of antigens from intestinal flora anaerobic bacteria reduces the severity of experimental acute colitis in BALB/c mice.” Clin. Exp. Immunol. 120: 46-50.

The entire disclosures of all documents cited throughout this application are incorporated herein by reference.

It will be appreciated by those skilled in the art that changes could be made to the embodiments described above without departing from the broad inventive concept described herein. It is understood, therefore that this invention is not limited to the particular embodiments and aspects disclosed, but it is intended to cover all modifications that are within the spirit and scope of the invention, as defined by the appended claims. Any combination of the above-described elements in all possible variations thereof is encompassed by the invention unless otherwise indicated herein or otherwise clearly contradicted by context.

What is claimed is:

1. A compound of the formula Ia: 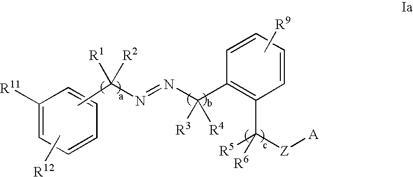
wherein:
A is hydrogen or is a residue of a hydroxyl-bearing drug;
Z is selected from the group consisting of —C(O)—, —S(O)—, —OC(O)—, —OC(O)NR¹³—, —S—C(O)—, —SC(O)NR¹³—, —C(O)NR¹³—, —NR¹³C(O)NR¹³—, —OS(O)—, —OS(O)₂—, —S(O)₂NH— and —OPO(OH)—;
each R¹, R², R⁵ and R⁶ is independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted;
each R³ and R⁴ is independently selected from the group consisting of hydrogen (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each substituted or unsubstituted;
R¹¹ is selected from the group consisting of hydrogen, hydroxy, (C₁₋₃)alkoxy, and —C(O)OR¹⁰;
each R⁹ and R¹² is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, —SO₃R¹³, —PO₃R¹³, (C₁₋₃)alkoxy, each alkyl, cycloalkyl, aryl and heteroaryl substituted or unsubstituted;
or R¹¹ and R¹² when substituted adjacent in the phenyl ring, are taken together form an optionally substituted heterocyclic ring;
R¹⁰ is hydrogen or (C₁₋₃)alkyl;
each R¹³ is independently hydrogen or (C₁₋₃)alkyl, and
each a, b and c is independently 0, 1 or 2;
or a pharmaceutically acceptable salt thereof, optionally in the form of a single stereoisomer or mixture of stereoisomers.

2. The compound claim 1, wherein R¹¹ and R¹² are taken together to form an optionally substituted heterocyclic ring.

3. The compound of claim 2, wherein R¹¹ and R¹² are taken together to form acetonidyl-4-one.

4. The compound of claim 1, wherein R¹¹ is —C(O)O—R¹⁰, and each of R¹² and R¹⁰ is hydrogen.

5. A compound of the formula I: 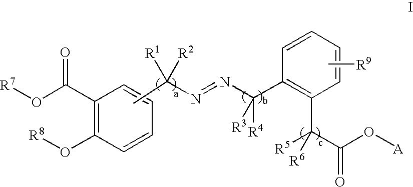
wherein:
A is hydrogen or is a residue of a hydroxyl-bearing drug;
each R¹, R², R⁵ and R⁶ is independently selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted;
each R³ and R⁴ are independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each substituted or unsubstituted;
R⁷ is hydrogen or is (C₁₋₃)alkyl;
R⁸ is hydrogen or is (C₁₋₃)alkyl;
R⁹ is selected from the group consisting of hydrogen, (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, each substituted or unsubstituted; and
each a, b and c is independently 0, 1 or 2;
or a pharmaceutically acceptable salt thereof, optionally in the form of a single stereoisomer or mixture of stereoisomers.

6. The compound of claim 5, wherein R⁷ and R⁸ are hydrogen, and c is 1 or 2.

7. The compound of claim 5, wherein a and b are both 0, and c is 1 or 2.

8. The compound of claim 5, wherein R⁷ and R⁸ are hydrogen, R⁹ is hydrogen, and a and b are both 0.

9. The compound of claim 5, wherein the hydroxyl-bearing drug is selected from the group consisting of an anti-inflammatory drug, an anti-cancer drug, an imaging agent; a vaccine, an antigen, an anti-infective drug, a peptide, an antisense molecule and a protein.

10. The compound of claim 9, wherein the hydroxyl-bearing drug is an anti-inflammatory drug.

11. The compound of claim 10, wherein the anti-inflammatory drug is a steroid.

12. The compound of claim 11, wherein the steroid is selected from the group consisting of hydrocortisone, dexamethasone, budesonide esterified at the 21-, 11- and 17-positions, respectively and prednisolone.

13. The compound of claim 9, wherein the hydroxyl-bearing drug is selected from the group consisting of nitroimidazoles, quinolines such as nalidixic acid, fluoroquinolones, ciprofloxacin, levofloxacin, aminoglycosides, amikacin, gentamicin, kanamycin, neomycin, netilmicin, paromomycin, streptomycin, tobramycin and apramycin, leucovorin, topotecan, irinotecan, methotrexate, bevacizumab, cetuximab, panitumumab and infliximab.

14. A compound of the formula II: 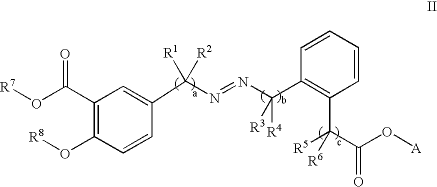
wherein:
A is hydrogen or is a residue of a hydroxyl-bearing drug;
each R¹, R², R⁵ and R⁶ are independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted;
each R³ and R⁴ are independently hydrogen or are each independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, amino, cyano, halo, hydroxy, (C₁₋₃)alkoxy, aryloxy and heteroaryloxy, each substituted or unsubstituted;
R⁷ is hydrogen or is (C₁₋₃)alkyl;
R⁸ is hydrogen or is (C₁₋₃)alkyl;
each a, b and c is independently 0, 1 or 2;
or a pharmaceutically acceptable salt thereof, optionally in the form of a single stereoisomer or mixture of stereoisomers.

15. The compound of claim 14, wherein the hydroxyl-bearing drug is selected from a group consisting of an anti-inflammatory drug, an anti-cancer drug, an imaging agent; a vaccine, an antigen, an anti-infective drug, a peptide, an antisense molecule and a protein.

16. The compound of claim 15, wherein the hydroxyl-bearing drug is an anti-inflammatory drug.

17. The compound of claim 16, wherein the anti-inflammatory drug is a steroid.

18. The compound of claim 17, wherein the steroid is selected from the group consisting of hydrocortisone, dexamethasone, budesonide esterified at the 21-, 11- and 17-positions, respectively, and prednisolone.

19. The compound of claim 15, wherein the hydroxyl-bearing drug is selected from the group consisting of nitroimidazoles, quinolines, fluoroquinolones, aminoglycosides, amikacin, gentamicin, kanamycin, neomycin, netilmicin, paromomycin, streptomycin, tobramycin, apramycin, leucovorin, topotecan, irinotecan, methotrexate, bevacizumab, cetuximab, panitumumab and infliximab.

20. A compound of the formula III: 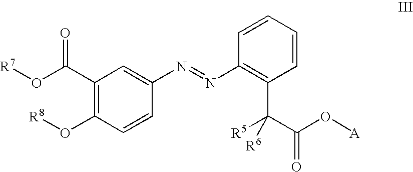
wherein:
A is hydrogen or is a residue of a hydroxyl-bearing drug;
R⁵ and R⁶ are independently hydrogen or are independently selected from the group consisting of (C₁₋₃)alkyl, (C₃₋₁₀)cycloalkyl, (C₃₋₁₀)cycloalkyl(C₁₋₃)alkyl, aryl, aryl(C₁₋₃)alkyl, heteroaryl, heteroaryl(C₁₋₃)alkyl, cyano, halo, hydroxy, (C₁₋₃)alkoxy, aryloxy and heteroaryloxy, each substituted or unsubstituted;
R⁷ is hydrogen or is (C₁₋₃)alkyl;
R⁸ is hydrogen or is (C₁₋₃)alkyl;
or a pharmaceutically acceptable salt thereof, optionally in the form of a single stereoisomer or mixture of stereoisomers.

21. The compound of claim 20, wherein R⁵ and R⁶ are hydrogen.

22. The compound of claim 20, wherein R⁵, R⁶, R⁷ and R⁸ each are hydrogen.

23. The compound of claim 20, wherein the hydroxyl-bearing drug is selected from a group consisting of an anti-inflammatory drug, an anti-cancer drug, an imaging agent; a vaccine, an antigen, an anti-infective drug, a peptide, an antisense molecule and a protein.

24. The compound of claim 23, wherein the hydroxyl-bearing drug is an anti-inflammatory drug.

25. The compound of claim 24, wherein the anti-inflammatory drug is a steroid.

26. The compound of claim 25, wherein the steroid is selected from the group consisting of hydrocortisone, dexamethasone, budesonide esterified at the 21-, 11- and 17-positions, respectively, and prednisolone.

27. The compound of claim 20, wherein A is selected from the group consisting of nitroimidazoles, quinolines, fluoroquinolones, aminoglycosides, gentamicin, kanamycin, neomycin, netilmicin, paromomycin, streptomycin, tobramycin and apramycin, leucovorin, topotecan, irinotecan, methotrexate, bevacizumab, cetuximab, panitumumab and infliximab.

28. The compound of claim 1, where the compound is selected from the group consisting of:

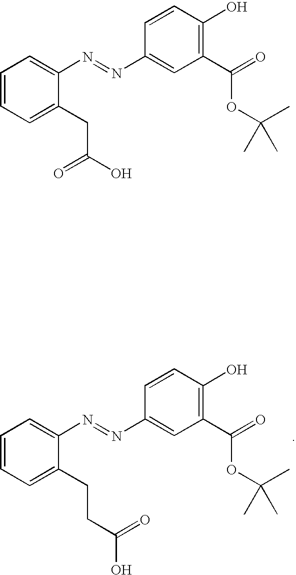

29. The compound of claim 1, where the compound is selected from the group consisting of:

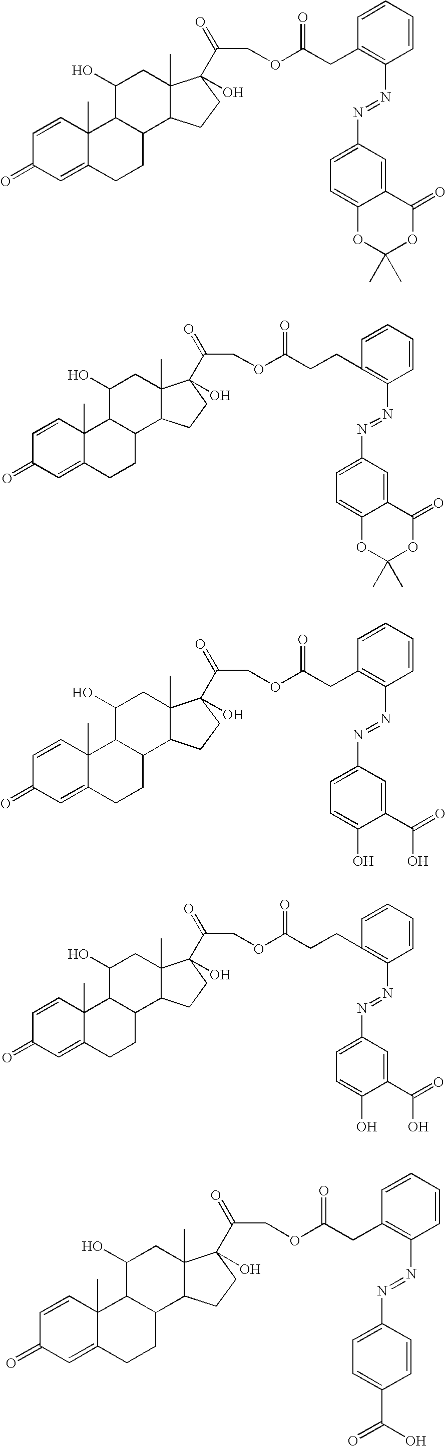

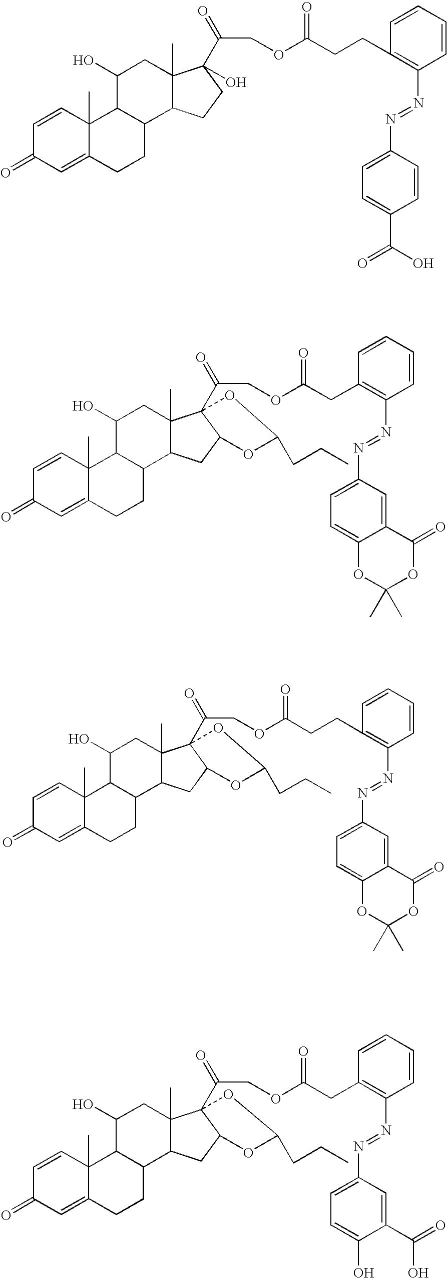

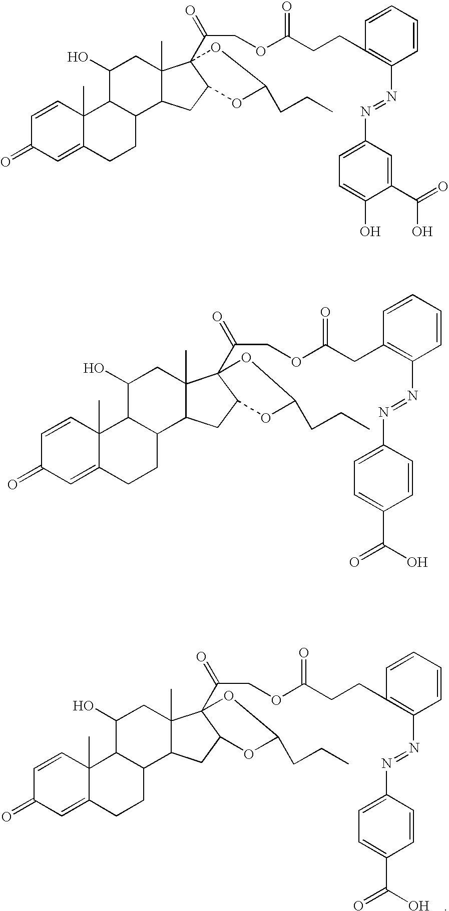

30. A pharmaceutical composition comprising a therapeutically effective amount of a compound of claim 20, and a pharmaceutically acceptable excipient.

31. A method for decreasing NFκB DNA-binding activity in a patient, the method comprising administering a therapeutically effective amount of a compound or composition of claim 30 to the patient.

32. The method of claim 31, wherein the therapeutically effective amount is effective to reduce, alleviate or treat inflammatory bowel disease (IBD).

33. The method of claim 31, wherein the therapeutically effective amount is effective to reduce, alleviate or treat ulcerative colitis or Crohn's disease.

34. A method of treating inflammatory bowel disease (IBD), comprising administering a therapeutically effective amount of a compound or composition of claim 30 to a mammal in need of such treatment.

35. A method of treating Crohn's disease or ulcerative colitis, comprising administering a therapeutically effective amount of a compound or composition of claim 30 to a mammal in need of such treatment.

36. The method of claim 35 wherein the amount of a compound or composition administered is effective to maintain remission.

37. The method of claim 36 wherein 5ASA-5ASA is administered.

38. A method of reducing, alleviating or treating acute ulcerative colitis comprising administering a therapeutically effective amount of 5ASA-steroid, wherein steroid is hydrocortisone, dexamethasone, budesonide esterified at the 21-, 11- and 17-positions, respectively, or prednisolone.

39. The method of claim 38 wherein the steroid is prednisolone.

40. A method of treating Collagenous colitis, Lymphocytic colitis, Ischaemic colitis, Diversion colitis, Behçet's syndrome, Infective colitis, or Indeterminate colitis comprising administering a therapeutically effective amount of a compound or composition of claim 30 to a mammal in need of such treatment.

41. A method of treating Amebiasis, Clostridium Difficile Infection, Pseudomembranous colitis, Diverticulitis, Gastroenteritis, Gastrointestinal Cancers, or Irritable Bowel Syndrome (IBS) comprising administering a therapeutically effective amount of a compound or composition of claim 30 to a mammal in need of such treatment.

42. A method for treating or alleviating an inflammatory condition in a mammal, the method comprising delivering an effective amount of a COX2 inhibitor to the colon, wherein the COX2 inhibitor is the residue of a hydroxyl bearing drug of the compound or compositions of claim 30.

43. A method for treating gastrointestinal cancer in a mammal, the method comprising delivering an effective amount of a COX2 inhibitor to the colon, wherein the COX2 inhibitor is the residue of a hydroxyl bearing drug of the compound or compositions of claim 30.
